(19) United States
(12) Patent Application Publication
(10) Pub. No.: US 2006/0088826 A1
(43) Pub. Date: Apr. 27, 2006

(54) Discrimination and detection of target nucleotide sequences using mass spectrometry

(76) Inventors: Michael Josephus Theresia Van Eijk, Herpen (NL); Catharinus Van Schaik, Macharen (NL)

Correspondence Address: MCKENNA LONG & ALDRIDGE LLP, 1900 K STREET, NW, WASHINGTON, DC, 20006, US

(21) Appl. No.: 10/500,109

(22) PCT Filed: Dec. 30, 2002
(86) PCT No.: PCT/NL02/00872

§ 371 Date: Jan. 12, 2005

(30) Foreign Application Priority Data
Dec. 28, 2001 (NL) 01205114.0

(51) Int. Cl. C12Q 1/68; C12P 19/34

(52) U.S. Cl. 435/6; 435/91.2

(57) ABSTRACT

A method for determining the presence or absence of at least one target sequence in a nucleic acid sample, comprising the steps of performing an oligonucleotide ligation assay, digesting the resulting amplified probes with a restriction enzyme isolating detectable fragments and the determining the presence of detectable fragments by mass spectrometry.

### FIELD OF THE INVENTION

The present invention relates to the field of biotechnology. In particular the present invention provides a method for the discrimination and detection of nucleotide sequences using a detection technique based on molecular mass. The invention further provides for the application of the method in the discrimination and identification of (multiple) target sequences that may contain single nucleotide polymorphisms. The invention also provides for oligonucleotide probes that are capable of hybridising to the target sequence of interest, primers for the amplification of ligated probes, use of these probes and primers in the identification and/or detection of nucleotide sequences that are related to a wide variety of genetic traits and genes and kits of primers and/or probes suitable for use in the method according to the invention.

### BACKGROUND OF THE INVENTION

There is a rapidly growing interest in the detection of specific nucleic acid sequences. This interest has not only arisen from the recently disclosed draft nucleotide sequence of the human genome and the presence therein, as well as in the genomes of many other organisms, of an abundant amount of single nucleotide polymorphisms (SNP), but also from marker technologies such as AFLP. The recognition that the presence of single nucleotide substitutions (and other types of genetic polymorphisms such as small insertion/deletions; indels) in genes provide a wide variety of information has also attributed to this increased interest. It is now generally recognised that these single nucleotide substitutions are one of the main causes of a significant number of monogenically and multigenically inherited diseases, for instance in humans, or are otherwise involved in the development of complex phenotypes such as performance traits in plants and livestock species. Thus, single nucleotide substitutions are in many cases also related to or at least indicative of important traits in humans, plants and animal species.

Analysis of these single nucleotide substitutions and indels will result in a wealth of valuable information, which will have widespread implications on medicine and agriculture in the widest possible terms. It is for instance generally envisaged that these developments will result in patient-specific medication. To analyse these genetic polymorphisms, there is a growing need for adequate, reliable and fast methods that enable the handling of large numbers of samples and large numbers of (predominantly) SNPs in a high throughput fashion, while at the same time maintaining the quality of the data.

Even though a wide diversity of detection platforms for SNPs exist at present (such as fluorometers, DNA microarrays, mass-spectrometers and capillary electrophoresis instruments), the major limitation to achieve cost-effective high throughput detection is that a robust and efficient multiplex amplification technique for non-random selection of SNPs is currently lacking to utilise these platforms efficiently, which results in suboptimal use of these powerful detection platforms and/or high costs per datapoint.

Specifically, using common amplification techniques such as the PCR technique it is possible to amplify a limited number of target sequences by combining the corresponding primer pairs in a single amplification reaction. However, the number of target sequences that can be amplified simultaneously is small and extensive optimisation may be required to achieve similar amplification efficiencies of the individual target sequences. One solution to multiplex amplification is to use a single primer pair for the amplification of all target sequences, which requires that all targets must contain the corresponding primer-binding sites. This principle is incorporated in the AFLP technique (EP-A 0 534 858). Using AFLP, the primer-binding sites result from a digestion of the target nucleic acid (i.e. total genomic DNA or cDNA) with one or more restriction enzymes, followed by adapter ligation. AFLP essentially targets a random selection of sequences contained in the target nucleic acid. It has been shown that, using AFLP, a practically unlimited number of target sequences can be amplified in a single reaction, depending on the number of target sequences that contain primer-binding region(s) that are perfectly complementary to the amplification primers. Exploiting the use of single primer-pair for amplification in combination with a non-random method for SNP target selection and efficient use of detection platforms may therefore substantially increase the efficiency of SNP genotyping, however such technology has not been provided in the art yet.

One of the principal methods used for the analysis of the nucleic acids of a known sequence is based on annealing two probes to a target sequence and, when the probes are hybridised adjacently to the target sequence, ligating the probes. The OLA-principle (Oligonucleotide Ligation Assay) has been described, amongst others, in U.S. Pat. No. 4,988,617 (Landegren et al). This publication discloses a method for determining the nucleic acid sequence in a region of a known nucleic acid sequence having a known possible mutation. To detect the mutation, oligonucleotides are selected to anneal to immediately adjacent segments of the sequence to be determined. One of the selected oligonucleotide probes has an end region wherein one of the end region nucleotides complementary to either the normal or to the mutated nucleotide at the corresponded position in the known nucleic acid sequence. A ligase is provided which covalently connects the two probes when they are correctly base paired and are located immediately adjacent to each other. The presence or absence of the linked probe is an indication of the presence of the known sequence and/or mutation.

U.S. Pat. No. 5,876,924 by Zhang et al. also describes a ligation reaction using adjacent probes wherein one of the probes is a capture probe with a binding element such as biotin. After ligation, the unligated probes are removed and the ligated captured probe is detected using paramagnetic beads with a ligand (biotin) binding moiety.

Abbot et al. in WO 96/15271 developed a method for a multiplex ligation amplification procedure comprising of the hybridisation and ligation of adjacent probes. These probes are provided with an additional length segment, the sequence of which, according to Abbot et al., is unimportant. The deliberate introduction of length differences intends to facilitate the discrimination on the basis of fragment length in gel-based techniques.

WO 97/45559 (Barany et al.) describes a method for the detection of nucleic acid sequence differences by using combinations of ligase detection reactions (LDR) and polymerase chain reactions (PCR). The LDR oligonucleotide probes in a given set may generate a unique length product and thus may be distinguished from other products based on size. WO 97/45559 discloses methods comprising annealing allele-specific probe sets to a target sequence and subsequent ligation with a thermostable ligase. Amplification of the ligated products with fluorescently labelled primers results in a fluorescently labelled amplified product. Detection of the products is based on separation by size or electrophoretic mobility or on an addressable array.

This method allows for the detection of a number of nucleic acid sequences in a sample. However, the design, validation and routine use of arrays for the detection of amplified probes involves many steps (ligation, amplification, optionally purification of the amplified material, array production, hybridisation, washing, scanning and data quantification), of which some (particularly hybridisation and washing) are difficult to automate. Array-based detection is therefore laborious and costly to analyse a large number of samples for a large number of SNPs.

The method and the various embodiments described by Barany et al. are found to have certain disadvantages. One of the major disadvantages is that the method in principle does not provide for a true high throughput process for the determination of large numbers of target sequences in short periods of time using reliable and robust methods without compromising the quality of the data produced and the efficiency of the process.

More in particular, one of the disadvantages of the means and methods as disclosed by Barany et al. resides in the limited multiplex capacity when discrimination is based inter alia, on the length of the allele specific probe sets. Discrimination between sequences that are distinguishable by only a relatively small length difference is, in general, not straightforward and carefully optimised conditions may be required in order to come to the desired resolving power. Discrimination between sequences that have a larger length differentiation is in general easier to accomplish. This may provide for an increase in the number of sequences that can be analysed in the same sample. However, providing for the necessary longer nucleotide probes is a further hurdle to be taken. In the art, synthetic nucleotide sequences are produced by conventional chemical step-by-step oligonucleotide synthesis with a yield of about 98.5% per added nucleotide. When longer probes are synthesized (longer than ca. 60 nucleotides) the yield generally drops and the reliability and purity of the synthetically produced sequence can become a problem.

Another disadvantage of the means and methods as disclosed by Barany et al. resides herein that for increasing the multiplex capacity of the method, the span (i.e. the difference between the shortest and the longest) length difference between the ligated products corresponding to different target sequences within a sample must increase. The use of a relatively large span within the amplifiable ligated products may result in differential amplification efficiencies in favour of the shorter sequences. This adversely affects the overall data quality, hampering the development of a true high throughput method. Thus the need for a reliable and cost-efficient solution to multiplex amplification and subsequent detection for high throughput application remains.

These and other disadvantages of the methods disclosed in WO 97/45559 lead the present inventors to the conclusion that the methods described therein are less preferable for adaptation in a high throughput protocol that is also capable of handling a large number of samples that may each comprise a large numbers of sequences.

Mass-spectroscopy techniques such as matrix assisted laser desorption/ionisation time-of-flight (MALDI-TOF) for detecting/identifying single strand

DNA fragments are known, for instance from WO 00/31300, WO 97/47766; WO 98/54571; WO 99/02728; WO 97/33000, as well as Griffin et al., Proc. Natl. Acad. Sci. USA., Vol. 96, pp. 6301-6306 (1999); Ross et al., Nature Biotechnology, Vol. 16 (1998), p. 1347-1351; and Berkenkamp et al., Science, Vol. 281 (1998), p. 260-262.

These techniques known in the art suffer from at least one major disadvantage, which is that the resolution for fragments with a larger mass is significantly lower than that for fragments with a relative small mass. Accordingly, reliable and reproducible detection of fragments with a large mass, for instance relatively long fragments such oligonucleotides ranging from ca. 50 nucleotides to more than 500, becomes cumbersome. As a consequence, detection of relatively long ligated products such as those obtained via the above-discussed oligonucleotide ligation assays, using mass detection is not a preferred route for the development of high throughput assays.

### DESCRIPTION OF THE INVENTION

The present invention provides for a method for determining the presence or absence of a target sequence in a nucleic acid sample, wherein the presence or absence of the target sequence is determined by an oligonucleotide ligation assay in combination with a detection method based upon molecular mass and wherein each target sequence in the sample is represented by a stuffer and detection of the target sequences is based on the detection of the presence or the absence of a fragment comprising said stuffer. The present invention thus provides a method for transferring the information on the occurrence of a ligation event and hence on the presence of a target sequence to a mass detectable stuffer.

### DETAILED DESCRIPTION OF THE INVENTION

In a first aspect the invention relates to a method for determining the presence or absence of a target sequence in a nucleic acid sample, wherein the presence or absence of the target sequence is determined by an oligonucleotide ligation assay in combination with a detection method based upon molecular mass and wherein each target sequence in the sample is represented by a stuffer and detection of the target sequences is based on the detection of the presence or the absence of a fragment comprising said stuffer.
A preferred aspect of the invention pertains to a method for determining the presence or absence of at least one target sequence (2) in a nucleic acid sample, comprising the steps of:
   a) providing to a nucleic acid sample a pair of a first and a second oligonucleotide probe for each target sequence to be detected in the sample, whereby the first oligonucleotide probe has a section (4) at its 5′-end that is complementary to a first part (5) of a target sequence and the second oligonucleotide probe has a section (6) at its 3′-end that is complementary to a second part (7) of the target sequence, whereby the first (5) and second part (7) of the target sequence are located adjacent to each other, and whereby the first and second oligonucleotide probes (4, 6) each comprise a tag sequence (8, 9), whereby the tag sequences are essentially non-complementary to the target sequence, whereby the tag sequences comprise primer-binding sequences (12, 13), and wherein at least one of the tags further comprises a stuffer (11) and a restriction site (10) for a restriction enzyme, which restriction site (10) is located between the primer binding site and the section of the oligonucleotide probe (4, 6) that is complementary to the first (5) or second part (7) of the target sequence and wherein the stuffer (11) is located between the restriction site (10) and the primer binding site;
   b) allowing the oligonucleotide probes to anneal to the adjacent parts of target sequence whereby the complementary sections (4,6) of the first and the second oligonucleotide probes are adjacent;
   c) providing means (14) for connecting the first and the second oligonucleotide probes annealed adjacently to the target sequence and allowing the complementary sections (4, 6) of the adjacently annealed first and second oligonucleotide probes to be connected, to produce a connected probe (15) corresponding to a target sequence in the sample;
   d) amplifying the connected probes from a primer pair (16, 17) to produce an amplified sample (19) comprising amplified connected probes (20);
   e) digesting the amplified connected probes with the restriction enzyme to produce a detectable fragment (21);
   f) detecting the presence or absence of the target sequence by detecting the presence or absence of the detectable fragment by a detection method based upon molecular mass.

In step a) a multiplicity of target sequences, or at least one, preferably at least two target sequence(s) is/are brought into contact with a corresponding multiplicity of specific oligonucleotide probes under hybridising conditions. The pairs of oligonucleotide probes are subsequently allowed to anneal to the adjacent complementary parts of the multiple target sequences in the sample.

Methods and conditions for specific annealing of oligonucleotide probes to complementary target sequences are well known in the art (see e.g. in Sambrook and Russel (2001) “Molecular Cloning: A Laboratory Manual (3^{rd} edition), Cold Spring Harbor Laboratory, Cold Spring Harbor Laboratory Press). Usually, after mixing of the oligonucleotide probes and target sequences the nucleic acids are denatured by incubation for a short period of time (e.g. 30 seconds to 5 minutes) in a low salt buffer (e.g. a buffer containing no salts or less salts than the ionic strength equivalent of 10 mM NaCl). The sample containing the denatured probes and target sequences is then allowed to cool to an optimal hybridisation temperature for specific annealing of the probes and target sequences, which usually is about 5° C. below the melting temperature of the hybrid between the complementary section of the probe and its complementary sequence (in the target sequence).

In order to prevent aspecific or inefficient hybridisation of one of the two probes in a primer pair, or in a sample with multiple target sequences, it is preferred that, within one sample, the sections of the probes that are complementary to the target sequences are of a similar, preferably identical melting temperatures between the different target sequences present in the sample. Thus, the complementary sections of the first and second probes preferably differ less than 20, 15, 10, 5, or 2° C. in melting temperature. This is facilitated by using complementary sections of the first and second probes with a similar length and similar G/C content. Thus, the complementary sections preferably differ less than 20, 15, 10, 5, or 2 nucleotides in length and their G/C contents differ by less than 30, 20, 15, 10, or 5%. Complementary as used herein means that a first nucleotide sequence is capable of specifically hybridising to second nucleotide sequence under normal stringency conditions.

A nucleotide sequence that is considered complementary to another nucleotide sequence may contain a minor amount, i.e. preferably less than 20, 15, 10, 5 or 2%, of mismatches. Alternatively, it may be necessary to compensate for mismatches e.g. by incorporation of so-called universal nucleotides, such as for instance described in EP-A 974 672, incorporated herein by reference. Since annealing of probes to target sequences is concentration dependent, annealing is preferably performed in a small volume, i.e. less than 10 pt. Under these hybridisation conditions, annealing of probes to target sequences usually is fast and does not to proceed for more than 5, 10 or 15 minutes, although a longer annealing time may be used as long as the hybridisation temperature is maintained to avoid aspecific annealing. To avoid evaporation during denaturation and annealing, the walls and lids of the reaction chambers (i.e. tubes or microtitre wells) may also be heated to the same temperature as the reaction mixture. In preferred oligonucleotide probes the length of the complementary section is preferably at least 15, 18 or 20 nucleotides and preferably not more than 30, 40, or 50 nucleotides and the probes preferably have a melting temperature of at least 50° C., 55° C. or 60° C.

In addition to the above hybridisation criteria, the complementary sections of the oligonucleotide probes are designed such that for each target sequence in a sample, a pair of a first and a second probe is provided, whereby the probes each contain a section at their extreme ends that is complementary to a part of the target sequence and the corresponding complementary parts of the target sequence are located essentially adjacent to each other.

Within a pair of oligonucleotide probes, the first oligonucleotide probe has a section at its 5′-end that is complementary to a first part of a target sequence and the second oligonucleotide probe has a section at its 3′-end that is complementary to a second part of the target sequence. Thus, when the pair of probes is annealed to complementary parts of a target sequence the 5′-end of the first oligonucleotide probe is essentially adjacent to the 3′-end of the second oligonucleotide probe such that the respective ends of the two probes may be ligated to form a phosphodiester bond.

The respective 5′- and 3′-ends of a pair of first and second oligonucleotide probes that are annealed essentially adjacent to the complementary parts of a target sequence are connected in step (c) to form a covalent bond by any suitable means known in the art. The ends of the probes may be enzymatically connected to form a phosphodiester bond by a ligase, preferably a DNA ligase. DNA ligases are enzymes capable of catalysing the formation of a phosphodiester bond between (the ends of) two polynucleotide strands bound at adjacent sites on a complementary strand. DNA ligases usually require ATP (EC 6.5.1.1) or NAD (EC 6.5.1.2) as a cofactor to seal nicks in double stranded DNA. Suitable DNA ligase for use in the present invention are T4 DNA ligase, E. coli DNA ligase or preferably a thermostable ligase like e.g. Thermus aquaticus (Taq) ligase, Thermus thermophilus DNA ligase, or Pyrococcus DNA ligase. Alternatively, chemical autoligation of modified polynucleotide ends may be used to ligate two oligonucleotide probes annealed at adjacent sites on the complementary parts of a target sequence (Xu and Kool, 1999, Nucleic Acid Res. 27: 875-881).

Both chemical and enzymatic ligation occur much more efficient on perfectly matched probe-target sequence complexes compared to complexes in which one or both of the probes form a mismatch with the target sequence at, or close to the ligation site (Wu and Wallace, 1989, Gene 76: 245-254; Xu and Kool, supra). In order to increase the ligation specificity, i.e. the relative ligation efficiencies of perfectly matched oligonucleotides compared to mismatched oligonucleotides, the ligation is preferably performed at elevated temperatures. Thus, in a preferred embodiment of the invention, a DNA ligase is employed that remains active at 50-65° C. for prolonged times, but which is easily inactivated at higher temperatures, e.g. used in the denaturation step during a PCR, usually 90-100° C. One such DNA ligase is a NAD requiring DNA ligase from a Gram-positive bacterium (strain MRCH 065) as known from WO 01/61033. This ligase is referred to as “Ligase 65” and is commercially available from MRC Holland, Amsterdam.

A preferred method of the invention further comprises a step for the removal of oligonucleotide probes that are not annealed to target sequences and/or that are not-connected/ligated. Removal of such probes preferably is carried out prior to amplification, and preferably by digestion with exonucleases.

By removal/elimination of the oligonucleotide probes that are not connected/ligated a significant reduction of ligation independent (incorrect) target amplification can be achieved, resulting in an increased signal-to-noise ratio. One solution to eliminate one or more of the not-connected/ligated components without removing the information content of the connected probes is to use exonuclease to digest not-connected/ligated oligonucleotide probes. By blocking the end that is not ligated, for example the 3′ end of the downstream oligonucleotide probe, one probe can be made substantially resistant to digestion, while the other is sensitive. Only the presence of full length ligation product sequence will then prevent digestion of the connected probe. Blocking groups include use of a thiophosphate group and/or use of 2-O-methyl ribose sugar groups in the backbone. Exonucleases include ExoI (3′-5′), Exo III (3′-5′), and Exo IV (both 5′-3′ and 3′-5′), the later requiring blocking on both sides. One convenient way to block both probes is by using one long “padlock” probe (see M. Nilsson et. al., “Padlock Probes: Circularising Oligonucleotides for Localised DNA Detection,” Science 265: 2085-88 (1994), which is hereby incorporated by reference), although this is by no means required.

An advantage of using exonucleases, for example a combination of Exo I (single strand specific) and Exo III (double strand specific), is the ability to destroy both the target DNA and one of the oligonucleotide probes, while leaving the ligation product sequences substantially undigested. By using an exonuclease treatment prior to amplification, either one or both (unligated) oligonucleotide probes in each set are substantially reduced, and thus hybridisation of the remaining oligonucleotide probes to the original target DNA (which is also substantially reduced by exonuclease treatment) and formation of aberrant ligation products which may serve as a suitable substrate for PCR amplification by the oligonucleotide primer set is substantially reduced.

The oligonucleotide probes further contain a tag that is essentially non-complementary to the target sequence. The tag does not or not significantly hybridise, preferably at least not under the above annealing conditions, to any of the target sequences in a sample, preferably not to any of the sequences or probes in the sample. The tag preferably comprises a primer-binding site and may optionally comprise a stuffer sequence of variable length (see below).

The connected probes are amplified using a pair of primers corresponding to the primer-binding sites. In a preferred embodiment at least one of the primers or the same set of primers is used for the amplification of two or more different connected probes in a sample, preferably for the amplification of all connected probes in a sample. The different primers that are used in the amplification in step (d) are preferably essentially equal in annealing and priming efficiency. Thus, the primers in a sample preferably differ less than 20, 15, 10, 5, or 2° C. in melting temperature. This can be achieved as outlined above for the complementary section of the oligonucleotide probes. Unlike the sequence of the complementary sections, the sequence of the primers is not dictated by the target sequence. Primer sequences may therefore conveniently be designed by assembling the sequence from tetramers of nucleotides wherein each tetramer contains one A,T,C and G or by other ways that ensure that the G/C content and melting temperature of the primers are identical or very similar. The length of the primers (and corresponding primer-binding sites in the tags of the probes) is preferably at least 12, 15 or 17 nucleotides and preferably not more than 25, 30, 40 nucleotides.

In step (d) of the method of the invention, the connected probes are amplified to produce a (detectable) amplified connected probe(s) by any suitable nucleic acid amplification method known in the art. Nucleic acid amplification methods usually employ two primers, dNTPs, and a (DNA) polymerase. A preferred method for amplification is PCR. “PCR” or “Polymerase Chain Reaction” is a rapid procedure for in vitro enzymatic amplification of a specific DNA segment. The DNA to be amplified is denatured by heating the sample. In the presence of DNA polymerase and excess deoxynucleotide triphosphates, oligonucleotides that hybridise specifically to the target sequence prime new DNA synthesis. One round of synthesis results in new strands of, in principle and depending on the length of the parental strands, indeterminate length, which, like the parental strands, can hybridise to the primers upon denaturation and annealing. The second cycle of denaturation, annealing and synthesis produces two single-stranded products that together compose a discrete double-stranded product, exactly the length between the primer ends. This discrete product accumulates exponentially with each successive round of amplification. Over the course of about 20 to 30 cycles, many million-fold amplification of the discrete fragment can be achieved. PCR protocols are well known in the art, and are described in standard laboratory textbooks, e.g. Ausubel et al., Current Protocols in Molecular Biology, John Wiley & Sons, Inc. (1995). Suitable conditions for the application of PCR in the method of the invention are described in EP-A 0 534 858 and Vos et al. (1995; Nucleic Acids Res. 23: 4407-4414), where multiple DNA fragments between 70 and 700 nucleotides and containing identical primer-binding sequences are amplified with near equal efficiency using one primer pair. Other multiplex and/or isothermal amplification methods that may be applied include e.g. ligase chain reaction (LCR), self-sustained sequence replication (3SR), Q-β-replicase mediated RNA amplification, rolling circle amplification (RCA) or strand displacement amplification (SDA). In some instances this may require replacing the primer-binding sites in the tags of the probes by a suitable (RNA) polymerase-binding site.

The process of probe hybridisation, ligation and amplification is outlined in FIG. 1, whereas the structure of an amplified connected probe is illustrated in FIG. 2.

In step (e) the amplified connected probes are cleaved or cut. Cleaving the amplified connected probes can be achieved by any suitable means known in the art as long as a reproducible cleaved or cut nucleotide strand is obtained. Reproducible in this respect refers to the preference that the means for cleaving or cutting cut the nucleotide sequence at the same position in the sequence of the amplified connected probes. The means for cleaving the amplified connected probe can be chemical or enzymatic, but are preferably enzymatic, such as a restriction enzyme. A preferred restriction enzyme is a restriction endonuclease. An amplified connected probe is preferably cleaved by the restriction enzyme at the restriction site that was provided in the tag of one of the probes. Cleaving the amplified connected probes produces either flush ends in which the terminal nucleotides of both strands resulting from the restriction step are base-paired, or staggered ends in which one of the ends resulting from the restriction step protrudes to give a (short) single strand extension. Preferably the restriction site is recognised by a sequence specific restriction endonuclease. In principle any restriction endonuclease known in the art can be used, as long as it produces a reproducible cut. Cleaving the amplified connected probes in the sample results in a detectable fragment.

Restriction endonucleases itself are widely known in the art. A suitable restriction enzyme can have a recognition sequence of 4, 5, 6, 7, or 8 or more nucleotides. Preferably the restriction endonuclease is a rare cutter, (i.e. has a recognition sequence of more than 4 nucleotides). Preferably the restriction enzyme is a type II enzyme. Preferred restriction enzymes are EcoRI, HindIII, BamHI. Other preferred restriction enzymes are 6-cutter restriction enzymes, preferably 6-cutters that are relatively inexpensive.

Cleavage of the amplified connected probes in step (e), for instance using a restriction endonucleases, results in detectable fragments (comprising the stuffer sequence) and the remains of the amplified connected probes (waste fragments) (FIG. 3). The waste fragments, comprise the ligated complementary sections (4,6). Digesting with a restriction endonuclease results in a detectable fragment which is double stranded. Both the detectable fragments and the waste fragments consist of two strands, one designated as the top strand and the other as the bottom strand. The detectable fragment can be subjected to a denaturation treatment to provide for the separate bottom strand and top strands. The bottom strand is essentially complementary to the top strand, i.e. the largest part of the nucleotide sequence of the top and bottom strand are complementary, with the exception of those nucleotides that are part of a staggered or sticky end, essentially as described herein-before and in FIG. 3. Either the top or the bottom strand can be detected, or both the top and the bottom strand.

Detection is based on the detection of the presence or absence of the detectable fragment. Detection of the detectable fragment is preferably indicative of the presence or absence of the amplified connected probes in the amplified sample and hence of the target nucleotide sequence in the nucleic acid sample. Preferably the detection is based on the detection of the top and/or the bottom strand of the detectable fragment. The detection of the bottom strand in addition to the top strand has the advantage that direct confirmation of the presence or absence of the target sequence is obtained in duplo.

The detection can be performed directly on the digested sample, but it is preferred that, prior to detection, the detectable fragment is isolated, purified or separated from the digested amplified connected probes. The detectable fragment can be isolated, purified or separated from the digested amplified connected probes by means known in the art such as spin column purification, reversed phase purification or, preferably by affinity labelling techniques such as a biotin-streptavidin combination, combined with a suitable carrier such as magnetic beads, probe sticks etc. Isolation, purification or separation can also be performed after a denaturation treatment on the top and/or bottom strands.

The detectable fragment is preferably labelled with an affinity label. The affinity label is preferably located at the extreme end of the detectable fragment, located distal from the restriction site or, after digestion, the remains of the restriction site. The top strand and/or the bottom strand of the detectable fragment can be equipped with the affinity label. Preferably it is the bottom strand that comprises the affinity label and the stuffer sequence. The notion top strand is generally used to indicate that the nucleotide sequence of the top strand at least in part corresponds to the part of the tag that comprises the stuffer, the restriction site and the primer binding site, i.e. the top strand contains a nucleotide sequence that is essentially identical to that of the probe. The bottom strand is the strand complementary to the top strand and is obtained after a first round of amplification by extension of a primer complementary to the primer binding site in the top strand and which primer is preferably equipped with an affinity label. Accordingly, the bottom strand contains a sequence that corresponds to the nucleotide sequence of one of the primers. In a particular preferred embodiment the bottom strand is equipped with the affinity label. Preferably the bottom strand is isolated from the sample comprising the denatured detectable fragments, preferably by the affinity label. Preferably it is the bottom strand that is detected using mass spectrometry. Hence detection of the bottom strand provides the information relating to the presence or the absence of the corresponding target nucleotide strand.

The affinity label can be used for the isolation of the top and/or the bottom strand from the mixture of digested amplified connected probes as schematically outlined in FIG. 3. As an affinity label, a biotin-streptavidin combination is preferred. The affinity labelled top strand, bottom strand or detectable fragment can subsequently be detected using detection techniques based on molecular mass.

As used herein, the term affinity label also encompasses affinity labels that are coupled via so-called ‘linkers’ (having a certain molecular mass) located between the nucleotide sequence of the tag and the actual affinity label.

In an alternative embodiment, the affinity label is provided in the tag that does not comprise the restriction site-stuffer combination (FIG. 2b). This allows for the isolation of the amplified connected probes prior to the digestion step. The resulting mixture, after restriction and optional denaturation, can directly be analysed using mass spectrometry. As the mass of the detectable fragments, or the top or bottom strands, is known or can at least be calculated, the waste fragments (i.e. the remains of the digested amplified connected probes) do not significantly compromise the detection as the detectable fragments, and both the top or bottom strands, are within a known and different mass range.

Detection techniques based on molecular mass are for instance mass spectrometry and more in particular the mass spectrometry techniques that are suitable for the detection of large molecules such as oligonucleotides. Examples of these techniques are matrix assisted laser desorption/ionisation time-of-flight (MALDI-TOF), BPLC-MS, GC-MS etcetera. Commonly the detection techniques based on molecular mass prefer that the submitted samples contain oligonucleotides in a single stranded form. In case the detectable fragment has been isolated as a double stranded oligonucleotide, the detectable fragment is preferably denatured, using techniques known in the art, to yield single stranded oligonucleotides for instance such as those described herein as top and/or bottom strands.

After digestion with a restriction endonuclease, the obtained detectable fragment preferably comprises a stuffer, remains of the restriction site, and the primer binding site. Optionally an affinity label can be attached to the top and/or the bottom strand, optionally via a linker. The mass to be detected hence is the summation of the molecular mass of the primer binding site, the stuffer, the remains of the restriction site and the optional affinity label and optional linker.

To distinguish between different target sequences in a nucleic acid sample, the detectable fragments are designed such that a detectable fragment corresponding to one target sequence in the sample differs in mass from a detectable fragment corresponding to another target sequence in the sample. Accordingly, a sample comprising multiple target sequences comprises (after ligation, amplification and digestion) multiple detectable fragments, each detectable fragment with a different mass. Upon denaturation of the detectable fragments in the respective top and bottom strands, the various top strands each have a different mass. Likewise, the various bottom strands each have a different mass. Preferably, the mass difference between two different detectable fragments (and hence between two top or bottom strands respectively) is provided by the difference in mass of the stuffer.

The top strand or the bottom strand can be regarded as comprising a constant section and a variable section. The constant section comprises the primer binding site, the optional affinity label (including the optional linker) and the remains of the restriction site. The variable section comprises the stuffer. The constant section is constant within one sample and is of a constant mass. The variable section preferably provides the difference in mass between strands that correspond to different target nucleotides in a sample

In one embodiment of the present invention, the detectable fragment (and consequently) the oligonucleotide probes are designed such that the constant section is also varied in mass. This allows for the creation of multiple regions within a mass spectrum. Each region will have a lower limit and an upper limit, thereby defining a window. The lower limit of the window is defined by the mass of the constant sequence. By using different constant sequences, different regions can be defined. Preferably, these regions do not overlap. Within one region a mass difference between the oligonucleotides to be detected is created by the mass difference between the stuffers essentially as described herein before. The upper limit of the region is at least the sum of the lower limit of the region and the stuffer with the largest mass. For example, two constant sections have a mass of 6489 Dalton and 8214, respectively. Stuffer sequences of up to two nucleotides provide for 15 different combinations (including the absence of a stuffer, hence mass 0), each with a different molecular weight, ranging from 0 up to 642 (AG or GA). This allows for two regions, one ranging from 6489 Dalton to 7131 Dalton and one region from 8214 Dalton to 8856 Dalton. This allows for an increase of the multiplex capacity of the present invention. This also allows for the pooling of samples prior to mass analysis. Both will increase the high throughput capacity of the present invention.

To design stuffers that can be used in the probes of the present invention and that are capable of providing a unique mass to every detectable fragment and hence the top strand or bottom strand in the sample, the stuffers preferably have to meet the following requirements: i) a limited number of identical consecutive bases to avoid slippage of the polymerase during the amplification step; ii) no internal recognition site for the restriction enzyme; iii) minimal mass difference to ensure adequate resolution; iv) no formation of hairpins, for instance with other parts of the ligation probes for instance due to intramolecular hybridisation.

Stuffers suitable for use in the invention can be designed using a method that computes all possible stuffer sequences up to a pre-determined length and that fulfil the criteria listed above (i-iv). This method can be performed using a computer program on a computer. This method can be considered as an invention in itself. The computer program can be provided on a separate data carrier such a as diskette. The method starts with providing the upper length limit of the stuffer sequence. The method subsequently calculates all possible permutations of nucleotide sequences and through a process of elimination and selection applies the criteria i-iii as listed herein-before. The number of allowable consecutive bases can be provided separately or can be predetermined. The recognition site for the restriction enzyme can be provided as separate input, but can also be derived from a database of known recognition sites for the restriction enzyme, depending on whether or not other the presence of recognition sequences of other restriction enzymes is allowed. The minimal mass difference can also be provided as separate input or as a predetermined parameter. The formation of hairpins can be checked by using a standard PCR-primer selection program such as Primer Designer version 2.0 (copyright 1990, 1991, Scientific and Educational software). The resulting stuffer sequences can be presented to the user in a suitable format, for instance on a data-carrier.

The method according to the invention allows for the analysis of a multiplicity of target sequences thereby significantly increasing the throughput of the number of samples that can be analysed. “Throughput” as used herein, defines a relative parameter indicating the number of samples and target sequences that can be analysed per unit of time.

In the nucleic acid sample, the nucleic acids comprising the target may be any nucleic acid of interest. Even though the nucleic acids in the sample will usually be in the form of DNA, the nucleotide sequence information contained in the sample may be from any source of nucleic acids, including e.g. RNA, polyA⁺ RNA, cDNA, genomic DNA, organellar DNA such as mitochondrial or chloroplast DNA, synthetic nucleic acids, DNA libraries, clone banks or any selection or combinations thereof. The DNA in the nucleic acid sample may be double stranded, single stranded, and double stranded DNA denatured into single stranded DNA. Denaturation of double stranded sequences yields two single stranded fragments one or both of which can be analysed by probes specific for the respective strands. Preferred nucleic acid samples comprise target sequences on cDNA, genomic DNA, restriction fragments, adapter-ligated restriction fragments, amplified adapter-ligated restriction fragments. AFLP fragments or fragments obtained in an AFLP-template preamplification.

In its widest definition, the target sequence may be any nucleotide sequence of interest. The target sequence preferably is a nucleotide sequence that contains, represents or is associated with a polymorphism. The term polymorphism herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, microsatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A diallelic polymorphism has two forms. A triallelic polymorphism has three forms. A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. Single nucleotide polymorphisms can also arise from a deletion of a nucleotide or an insertion of a nucleotide relative to a reference allele. Other polymorphism include small deletions or insertions of several nucleotides, referred to as indels.

It is preferred that a sample contains two or more different target sequences, i.e. two or more refers to the identity rather than the quantity of the target sequences in the sample. In particular, the sample comprises at least two different target sequence, in particular at least 10, preferably at least 25, more preferably at least 50, more in particular at least 100, preferably at least 250, more preferably at least 500 and most preferably at least 1000 additional target sequences. In practice, the number of target sequences is limited, among others, by the number of connected probes. E.g., too many different pairs of first and second oligonucleotide probes in a sample may corrupt the reliability of the multiplex amplification step.

A further limitation is formed e.g. by the number of fragments in a sample that can be resolved by the detection device used in the present invention. The number can also be limited by the genome size of the organism or the transcriptome complexity of a particular cell type from which the DNA or cDNA sample, respectively, is derived. Detection in the present invention is based on mass-differences. State of the art mass-spectrometry allows for detection of mass differences below 1 Dalton. However, it is preferred that the mass difference between fragments (detectable fragments, bottom strands, top strands or stuffers) that are detected using the method according to the invention is more than 1, Dalton, preferably more than 5, 10, 15, 20, 25, 30, or 50 Dalton.

For each target sequence for which the presence or absence in a sample is to be determined, a specific pair of first and second oligonucleotide probes is designed with sections that are complementary to the adjacent first and second parts of each target sequence as described above. Thus, in the method of the invention, for each target sequence that is present in a sample, a corresponding (specific) amplified connected probe may be obtained in the amplified sample. Preferably, a multiplicity of first and second oligonucleotide probes complementary to a multiplicity of target sequences in a sample is provided. A pair of first and second oligonucleotide probes for a given target sequence in a sample will at least differ in nucleotide sequence from probe pairs for other target sequences, and will preferably also differ in mass from probe pairs for other targets, more preferably a probe pair for a given target will produce a connected probe and/or amplified connected probe that differs in mass from connected probes corresponding to other targets in the sample as described below. Preferably this difference in mass is provided by a stuffer with a different mass.

The probes that are not complementary to a part of the target sequence or that contain too many mismatches will not or only to a reduced extent hybridise to the target sequence when the sample is submitted to hybridisation conditions. Accordingly ligation is less likely to occur. The number of spurious ligation products from these probes in general will therefore not be sufficient and much smaller than the bona fide ligation products such that they are outcompeted during subsequent multiplex amplification. Consequently, they will not be detected or only to a minor extent.

The tag of the oligonucleotide probes may further comprise a stuffer sequence of a variable mass. The length of the stuffer varies from 0 to 500, preferably from 0 to 100, more preferably from 1 to 50 nucleotides. The length of the tag varies from 15 to 540, preferably from 18 to 140, more preferably from 20 to 75 nucleotides.

Preferably, the mass difference is provided by the mass of the stuffer sequence(s) in the oligonucleotide probes. By including in each oligonucleotide probe a stuffer of a pre-determined mass, the length of each amplified connected probe in an amplified sample can be controlled such that an adequate discrimination based on mass differences of the detectable fragment obtained in step (e) is enabled.

The mass differentiation between the detectable fragments obtained from target sequences in the sample is preferably chosen such that the detectable fragments can be distinguished based on their mass. This is accomplished by using stuffer sequences that result in distinguishable mass differences. Thus, from the perspective of resolving power, the mass differences between the different detectable fragment derived from the amplified connected probes, as may be caused by their stuffers, are as large as possible. However, for several other important considerations, as noted before, the length differences between the different amplified connected probes is preferably as small as possible: (1) the upper limit that exists in practice with respect to the length of chemically synthesized probes of about 100-150 bases at most; (2) the less efficient amplification of larger fragments; and (3) the increased chances for differential amplification efficiencies of fragments with a large length variation which works best with fragments in a narrow mass range. Preferably the mass differences between the sequences to be determined and provided by the stuffers is at least sufficient to allow discrimination between essentially all detectable fragments obtained by digesting amplified connected probes. By definition, based on chemical, enzymatic and biological nucleic acid synthesis procedures, the minimal useable size difference between different amplified connected probes in an amplified sample is one base, and this size difference fits within the resolving power of most mass spectrometry devices, especially in the lower size ranges. Thus based on the above it is preferred to use multiplex assays with amplification products wherein the mass difference between the detectable fragments and hence the top and bottom strands associated with the amplification products is caused by a minimum number of bases as possible, taking into account the other requirements for the design of stuffers as described herein before.

The connected probes obtained from the ligation of the adjacent first and second probes are amplified in step (d), using a primer set, usually consisting of a pair of primers for each of the connected probes in the sample. The primer pair comprises primers that are complementary to primer-binding sequences that are present in the connected probes, preferably at the respective 3′ and 5′ ends of the connected probes. A primer pair usually comprises a first and at least a second primer, but may consist of only a single primer that primes in both directions.

In a preferred embodiment, at least one of the first and second oligonucleotide probes that are complementary to at least two different target sequences in a sample comprise a tag sequence that comprises a primer-binding site that is complementary to a single primer sequence. Thus, preferably at least one of the first and second primer in a primer pair is used for the amplification of connected probes corresponding to at least two different target sequences in a sample, more preferably for the amplification of connected probes corresponding to all target sequences in a sample. Preferably only a single first primer is used and in some embodiments only a single first and a single second primer is used for amplification of all connected probes. Using common primers for amplification of multiple different fragments usually is advantageous for the efficiency of the amplification step.

Multiple sets of primers can be used, for instance primer with a different length, to further increase the multiplex capacity of the method. These primers can also be used for increasing the mass of the constant regions in the detectable fragments, essentially as described herein before.

xxIn a particular preferred embodiment, one or more of the primers used in the amplification step of the present invention is a selective primer. A selective primer is defined herein as a primer that, in addition to its universal sequence which is complementary to a primer binding site in the probe, contains a region that comprises so-called “selective nucleotides”. The region containing the selective nucleotides is located at the 3′-end of the universal primer.

The principle of selective nucleotides is disclosed inter alia in EP534858 and in Vos et al., Nucleic Acid Research, 1995, vol. 23, 4407-44014. The selective nucleotides are complementary to the nucleotides in the (ligated) probes that are located adjacent to the primer sequence. The selective nucleotides generally do not form part of the region in the (ligated) probes that is depicted as the primer sequence. Primers containing selective nucleotide are denoted as +N primers, in which N stands for the number of selective nucleotides present at the 3′-end of the primer. N is preferably selected from amongst A, C, T or G.

N may also be selected from amongst various nucleotide alternatives, i.e. compounds that are capable of mimicking the behaviour of ACTG-nucleotides but in addition thereto have other characteristics such as the capability of improved hybridisation compared to the ACTG-nucleotides or the capability to modify the stability of the duplex resulting from the hybridisation. Examples thereof are PNAs, LNAs, inosine etc. When the amplification is performed with more than one primer, such as with PCR using two primers, one or both primers can be equipped with selective nucleotides. The number of selective nucleotides may vary, depending on the species or on other particulars determinable by the skilled man. In general the number of selective nucleotides is not more than 10, but at least 5, preferably 4, more preferably 3, most preferred 2 and especially preferred is 1 selective nucleotide.

A +1 primer thus contains one selective nucleotide, a +2 primer contains 2 selective nucleotides etc. A primer with no selective nucleotides (i.e. a conventional primer) can be depicted as a +0 primer (no selective nucleotides added). When a specific selective nucleotide is added, this is depicted by the notion +A or +C etc.

By amplifying a set of (ligated) probes with a selective primer, a subset of (ligated) probes is obtained, provided that the complementary base is incorporated at the appropriate position in the desired of the probes that are supposed to be selectively amplified using the selective primer. Using a +1 primer, for example, the multiplex factor of the amplified mixture is reduced by a factor 4 compared to the mixture of ligated probes prior to amplification. Higher reductions can be achieved by using primers with multiple selective nucleotides, i.e. 16 fold reduction of the original multiplex ration is obtained with 2 selective nucleotides etc.

When an assay is developed which, after ligation, is to be selectively amplified, it is preferred that the probe contains the complementary nucleotide adjacent to the primer binding sequence. This allows for pre-selection of the ligated probe to be selectively amplified.

The use of selective primers in the present invention has proven to be advantageously when developing ligation based assays with high multiplex ratios of which subsequently only a specific part needs to be analysed resulting in further cost reduction of the ligation reaction per datapoint. By designing primers together with adjacent selective nucleotides, the specific parts of the sample that are to be amplified separately can be selected beforehand.

One of the examples in which this is useful and advantageous is in case of analysis of samples that contain only minute amounts of DNA and/or for the identification of different (strains of) pathogens. For example, in an assay directed to the detection of various strains of anthrax (Bacillus anthracis), for each of the strains a set of representative probes is designed. The detection of the presence or absence of this set (or a characterising portion thereof) of ligated probes after the hybridisation and ligation steps of the method of the invention may serve as an identification of the strain concerned. The selective amplification with specifically designed primers (each selective primer is linked to a specific strain) can selectively amplify the various strains, allowing their identification. For instance, amplification with an +A primer selectively amplifies the ligated probes directed to strain X where a +G primer selectively amplifies the ligated probes directed to strain Y. If desired, for instance in the case of small amounts of sample DNA, an optional first amplification with a +0 primer will increase the amount of ligated probes, thereby facilitating the selective amplification.

For example, a universal primer of 20 nucleotides becomes a selective primer by the addition of one selective nucleotide at its 3′-end, the total length of the primer now is 21 nucleotides. Alternatively, the universal primer can be shortened at its 5′-end by the number of selective nucleotides added. For instance, adding two selective nucleotides at the 3′-end of the primer sequence can be combined with the absence (or removal) of two nucleotides from the 5′end of the universal primer, compared to the original universal primer. Thus a universal primer of 20 nucleotides is replaced by a selective primer of 20 nucleotides. These primers are depicted as ‘nested primers’. The use of selective primers based on universal primers has the advantage that amplification parameters such as stringency and temperatures may remain essentially the same for amplification with different selective primers or vary only to a minor extent. Preferably, selective amplification is carried out under conditions of increased stringency compared to non-selective amplification. With increased stringency is meant that the conditions for annealing the primer to the ligated probe are such that only perfectly matching selective primers will be extended by the polymerase used in the amplification step. The specific amplification of only perfectly matching primers can be achieved in practice by the use of a so-called touchdown PCR profile wherein the temperature during the primer annealing step is stepwise lowered by for instance 0.5° C. to allow for perfectly annealed primers. Suitable stringency conditions are for instance as described for AFLP amplification in EP 534858 and in Vos et al., Nucleic Acid Research, 1995, vol. 23, 4407-44014. The skilled man will, based on the guidance find ways tot adapt the stringency conditions to suit his specific need without departing from the gist of the invention.

One of the further advantages of the selective amplification of ligated probes is that an assay with a high multiplex ratio can be adapted easily for detection with methods or on platforms that prefer a lower multiplex ratio. More in particular, the advantage associated with the use of selective primers in the method of the present invention is that the ligation step can be performed at a very high multiplex ratio whereas the detection technologies based on mass spectrometry such as described herein in general do not have sufficient capacity to adequately deal with highly multiplexed samples. There is no indication in the art that the increase in the multiplex ratio of mass based detection will increase to the same or comparable extent or at the same or comparable speed compared to the increase in the multiplex ratio of ligation based assays. Therefore amplification with selective primers as disclosed herein provides a solution to the problem of combining high multiplex ratio technology with low multiplex ratio technology.

Preferably the range of lengths of amplified connected probes in an amplified sample has a lower limit of 40, 60, 80, or 100 and an upper limit of 120, 140, 160, or 180 nucleotides, bases or base pairs. It is particularly preferred that the range of lengths of the amplified connected probes varies from 80 to 140 nucleotides. However, these number are strongly related to the current limits of the presently known techniques. Based on the knowledge provided by this invention, the skilled artisan is capable of adapting these parameters when other circumstances apply.

The reliability of the multiplex amplification is further improved by limiting the variation in the length of the amplified connected probes. Limitations in the length variation of amplified connected probes is preferred as it results in reduction of the preferential amplification of smaller amplified connected probes in a competitive amplification reaction with larger connected probes.

One of the most advantageous aspects of the present invention lies in the combination of multiplex ligation, multiplex amplification, preferably with a single primer pair or with multiple primer pairs which each amplify multiple connected probes, and multiplex detection of fragments of a different molecular mass. This allows for a significant improvement of the efficiency of the analysis of target sequences as well as a significant reduction in the costs for each target analysed over presently known technology.

One aspect of the invention pertains to the use of the method in a variety of applications. Application of the method according to the invention is found in, but not limited to, techniques such as genotyping, transcript profiling, genetic mapping, gene discovery, marker assisted selection, seed quality control, hybrid selection, QTL mapping, bulked segregant analysis, DNA fingerprinting and microsatellite analysis. Another aspect pertains to the simultaneous high throughput detection of the quantitative abundance of target nucleic acids sequences.

### Detection of Single Nucleotide Polymorphisms

One particular preferred application of the method according to the invention is found in the detection of single nucleotide polymorphisms (SNPs). A first oligonucleotide probe comprises a part that is complementary to a part of the target sequence that is preferably located adjacent to the polymorphic site, i.e. the single polymorphic nucleotide. A second oligonucleotide probe is complementary to the part of the target sequence such that its terminal base is located at the polymorphic site, i.e. is complementary to the single polymorphic nucleotide. If the terminal base is complementary to the nucleotide present at the polymorphic site in a target sequence, it will anneal to the target sequence and will result in the ligation of the two probes. When the end-nucleotide, i.e. the allele-specific nucleotide does not match, no ligation or only a low level of ligation will occur and the polymorphism will remain undetected.

When one of the target sequences in a sample is derived from or contains a single nucleotide polymorphism (SNP), in addition to the probes specific for that allele, further probes can be provided that not only allow for the identification of that allele, but also for the identification of each of the possible alleles of the SNP (co-dominant scoring). To this end a combination of types of probes can be provided: one type probe that is the same for all alleles concerned and one or more of the other type of probe which is specific for each of the possible alleles. These one or more other type of probes contain the same complementary sequence but differ in that each contains a nucleotide, preferably at the end, that corresponds to the specific allele. The allele specific probe can be provided in a number corresponding to the number of different alleles expected. The result is that one SNP can be characterised by the combination of one type of probe with four other type (allele-specific) probes, identifying all four theoretically possible alleles (one for A, T, C, and G), by incorporating stuffer sequences of different mass into the allele specific probes.

When detecting polymorphisms it is preferred that the difference in length between two or more (SNP) alleles of the polymorphism is not more than two, thereby ensuring that the efficiency of the amplification is similar between different alleles or forms of the same polymorphism.

In a particular embodiment, preferably directed to the identification of single nucleotide polymorphisms, the first oligonucleotide probe is directed to a part of the target sequence that does not contain the polymorphic site and the second oligonucleotide probe contains, preferably at the end distal from the primer-binding sequence, one or more nucleotide(s) complementary to the polymorphic site of interest. After ligation of the adjacent probes, the connected probe is specific for one of the alleles of a single nucleotide polymorphism. The stuffer sequence contained in the detectable fragment is preferably indicative of the allele that is to be analysed.

To identify the allele of polymorphic site in the target sequence, a set of oligonucleotide probes can be provided wherein one first probe is provided and one or more second probes. Each second probe then contains a specific nucleotide at the end of the complementary sequence, preferably the 3′-end, in combination with a known mass of the stuffer, see also FIG. 5. For instance, in case of an A/C polymorphism, the second probe can contain a specific nucleotide T in combination with a stuffer length of 3 nucleotides (CCC 867.6 Dalton) and another second probe for this polymorphism combines a specific nucleotide G with a stuffer of mass 906.6 Dalton (TAC). As the constant region (primer, the remains of the restriction site, and the biotin label) is preferably the same mass, this creates a mass difference of 39 Dalton. In case the presence and/or the abundance of all four theoretically possible nucleotides of the polymorphic site is desired, the stuffer-specific nucleotide combination can be adapted accordingly. In this embodiment, the number of nucleotides defines a region with a lower and an upper mass limit. It can be considered that the locus-specific information is coupled to the length of the stuffer and the allele-specific information of the polymorphic site is coupled to the mass of the stuffer. The combination length/mass of the stuffers can then be seen as indicative of the locus-allele combination. In a sample containing multiple target sequences, amplified with the same pair of amplification-primers or with multiple pairs of amplification primers, the stuffers can be chosen such that all top strands, bottom strands or detectable fragments are of a unique length. In FIG. 4 an illustration of this principle is provided of two loci and for each locus two alleles. In a preferred embodiment this principle can be extended to at least ten loci with at least two alleles per locus.

### Detection of Specific Target Sequence

The target sequence contains a known nucleotide sequence derived from a genome. Such a sequence does not necessarily contain a polymorphism, but is for instance specific for a gene, a promoter, an introgression segment or a transgene or contains information regarding a production trait, disease resistance, yield, hybrid vigor, is indicative of tumours or other diseases and/or gene function in humans, animals and plants. To this end, the complementary parts of the first probe and the second probe are designed to correspond to a, preferably unique, target sequence in genome, associated with the desired information. The complementary parts in the target sequence are located adjacent to each other. In case the desired target sequence is present in the sample, the two probes will anneal adjacently and after ligation, amplification and digestion can be detected.

In another aspect the present invention pertains to a nucleic acid probe comprising a part that is capable of hybridising to part of a target sequence and further comprising a primer-binding sequence, a restriction site and a stuffer. The part that is capable of hybridising to part of a target sequence and the primer binding site are located at the extreme ends of the nucleic acid probe. Preferably the restriction site is located between the part that is capable of hybridising to part of a target sequence and the primer binding site. Preferably the stuffer is located between the restriction site and the primer binding site.

The invention also pertains to a set of probes comprising of two or more probes wherein each probe comprises a part that is complementary to part of a target sequence and wherein the complementary parts of the probes are located essentially adjacent on the target sequence and wherein at least one of the probes further comprises a stuffer, which stuffer is located essentially next to the complementary part and a primer-binding sequence located essentially adjacent to the stuffer and a restriction site located essentially between the stuffer and the complementary part.

The invention in a further aspect, pertains to the use of a set of probes in the analysis of at least one nucleotide sequence and preferably in the detection of a single nucleotide polymorphism, wherein the set further comprises at least one additional probe that contains a nucleotide that is complementary to the known SNP allele. Preferably the set comprises a probe for each allele of a specific single nucleotide polymorphism. The use of a set of probes is further preferred in a method for the high throughput detection of single nucleotide polymorphisms.

Another aspect of the invention relates to the primers and more in particular to the set of primers used in the amplification step of the present invention.

The present invention also finds embodiments in the form of kits. Kits according to the invention are for instance kits comprising probes suitable for use in the method as well as a kit comprising primers, further a combination kit, comprising primers and probes, preferably all suitably equipped with enzymes buffers, etcetera, is provided by the present invention.

Another aspect of the present invention pertains to a method and an arrangement for the selection of nucleotide sequences of a specific mass, in particular for use as stuffers as described in the present application.

### DESCRIPTION OF THE FIGURES

FIG. 1: Oligonucleotide ligation assay: Providing oligonucleotide probes, each containing a section (4, 6) that is capable of annealing to complementary sections (5, 7) of the target sequence (2), followed by ligation of the adjacent sections of the probes to provide connected probes (15) and amplification of the connected probes from a primer pair (16, 17), one of which is biotinylated (17), to provide an amplified sample (19) comprising double stranded amplified connected probes (20).

FIG. 2: (a): Double stranded amplified connected probes, consisting of a top strand and a bottom strand, comprising a forward primer (16), a restriction site (10), a stuffer (11), a reverse primer binding site (17) and a biotin affinity label; (b) double stranded amplified connected probe with the biotin affinity label located at the forward primer (16).

FIG. 3: Digestion of the double stranded amplified connected probes at the restriction site using a restriction enzyme provides for a detectable fragment (21) and waste. After digestion, the biotinylated detectable fragments can be denatured to provide a top and bottom strand followed by purification of the bottom strand with streptavidin labelled paramagnetic beads or the biotinylated fragments can be purified using the biotin affinity label in combination with a streptavidin coated paramagnetic bead after which a denaturation step provides the bottom strand. Either way the bottom strand can be detected using mass spectrometry.

FIG. 4: SNP identification wherein the allele specific probe contains a stuffer and a restriction site. The connected probes (with the annealed primers) each have a different mass, representative of a ligation event. After amplification from the primer pair and digestion with a restriction enzyme, the detection of the fragments comprising the stuffer sequences identifies the SNP.

FIG. 5: SNP-detection in Arabidopsis: Mass spectrometric analysis of the Colombia sample (FIG. 5A) and of the Landsberg sample (FIG. 5B).

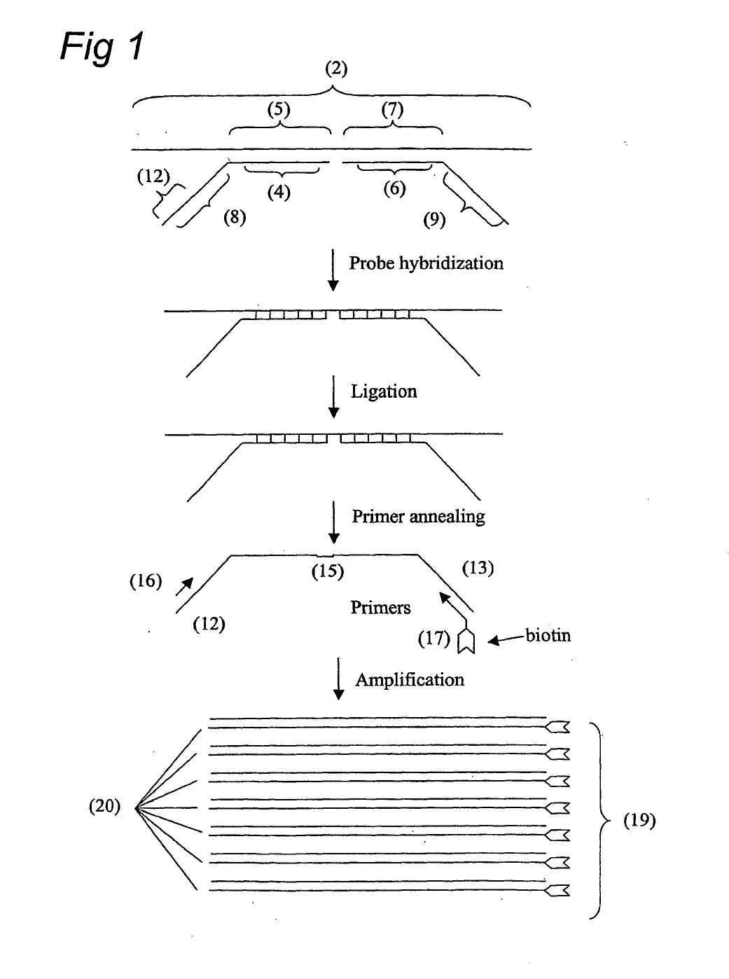

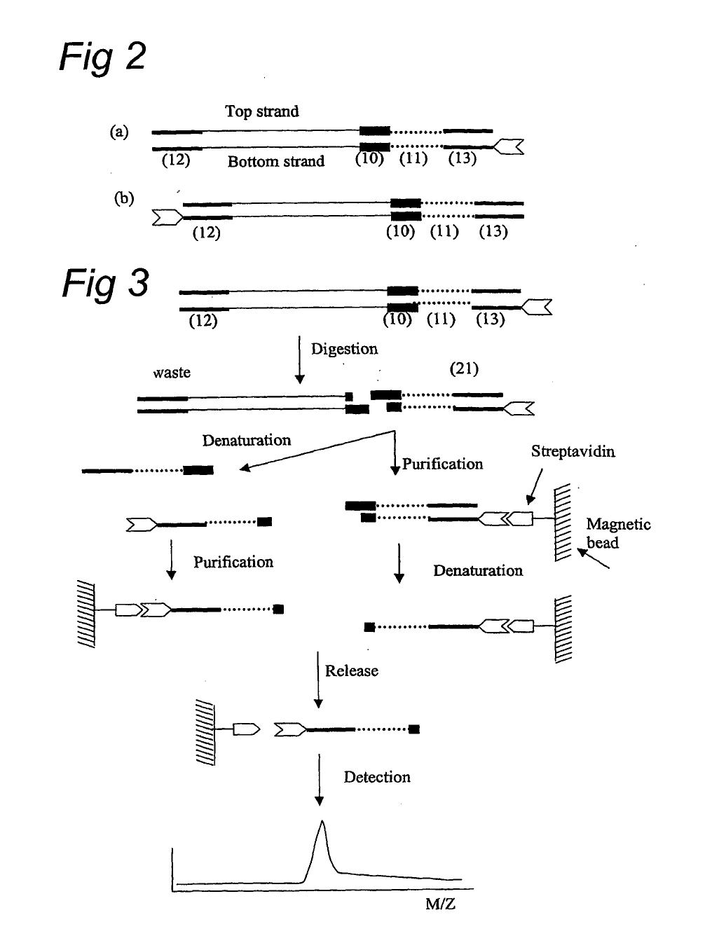

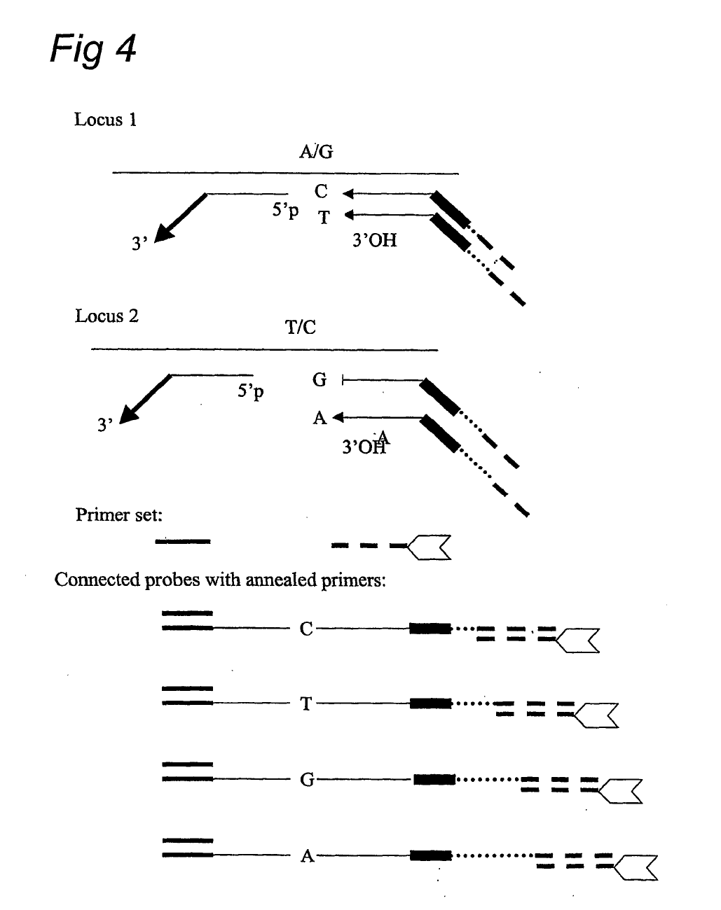

### EXAMPLES

### Example 1

### Description of Biological Materials and DNA Isolation

Recombinant Inbred (RI) lines generated from a cross between the Arabidopsis ecotypes Colombia and Landsberg erecta (Lister and Dean, 1993, Plant Journal 4, 745-750) were used the experiments described in Examples 6-10. Seeds from the parental and R1 lines were obtained from the Nottingham Arabidopsis Stock Centre. DNA was isolated from leaf material of individual seedlings using methods known per se, for instance essentially as described in EP-0534858, and stored in 1×TE (10 mM Tris-HCl pH 8.0 containing 1 mM EDTA) solution. Concentrations were determined by UV measurements in a spectrophotometer using standard procedures, and adjusted to 100 ng/μl using 1×TE.

### Example 2

### Selection of Arabidopsis SNP's

The Arabidopsis SNP's that were selected from The Arabidopsis Information Resource (TAIR) website: http://www.arabidopsis.org/SNPs.html:, are summarised in Table 1.

TABLE 1
Selected SNPs from Arabidopsis thaliana.

| | SNP | SNP alleles* | RI Map position |
|---|---|---|---|
| 1 | SGCSNP1 | G/A | chr. 2; 72,81 |
| 2 | SGCSNP20 | A/C | chr. 4; 15,69 |
| 3 | SGCSNP27 | T/G | chr. 3; 74,81 |
| 4 | SGCSNP37 | C/G | chr 2; 72,45 |
| 5 | SGCSNP39 | T/C | chr. 5; 39,64 |
| 6 | SGCSNP44 | A/T | not mapped |
| 7 | SGCSNP55 | C/A | chr. 5; 27,68 |
| 8 | SGCSNP69 | G/A | chr. 1; 81,84 |
| 9 | SGCSNP119 | A/T | chr. 4; 62,06 |
| 10 | SGCSNP164 | T/C | chr. 5; 83,73 |
| 11 | SGCSNP209 | C/G | chr. 1; 70,31 |
| 12 | SGCSNP312 | G/T | chr. 4; 55,95 |

For all SNP's the allele preceding the backslash is the Colombia allele.

### Example 3

### Oligonucleotide Probe Design for Oligonucleotide Ligation Reaction Selection of Stuffer Sequences

The stuffer sequences are selected from a total of 62 possible stuffer sequences that were calculated using the software program “Stuffer Selector” (Keygene N.V., Wageningen, The Netherlands), and meet the following criteria: minimal mass difference between the stuffers of 30 Dalton, a length of maximum 10 bases, no internal EcoRI site and no identical consecutive bases longer than 3 bases; see Table 3, output from “Stuffer Selector”

TABLE 3
Sixty-two stuffer sequences selected by the program “Stuffer Selector”.
Stuffer sequences were selected to have a mass difference of 30 Dalton,
a length of maximum of 8 bases, no runs of more than 3 identical bases
and no internal EcoRI site. Output from the software program “Stuffer
Selector”. Mass is in Dalton. Stuffers generated for
restriction enzyme: EcoRI

| Stuffer no. | Mass Stuffer | Sequence | [SEQ ID NO]. |
|---|---|---|---|
| 1 | 289.2 | C | |
| 2 | 329.2 | G | |
| 3 | 578.4 | CC | |
| 4 | 617.4 | TA | |
| 5 | 658.4 | GG | |
| 6 | 867.6 | CCC | |
| 7 | 906.6 | TAC | |
| 8 | 937.6 | TGT | |
| 9 | 971.6 | AGG | |
| 10 | 1171.8 | CCTC | 1 |
| 11 | 1204.8 | ACCA | 2 |
| 12 | 1235.8 | CATG | 3 |
| 13 | 1266.8 | TTGG | 4 |
| 14 | 1300.8 | GGAG | 5 |
| 15 | 1461.0 | CTCCC | 6 |
| 16 | 1494.0 | CCACA | 7 |
| 17 | 1525.0 | TAGCC | 8 |
| 18 | 1556.0 | CTGGT | 9 |
| 19 | 1589.0 | GATGA | 10 |
| 20 | 1621.0 | GGGTG | 11 |
| 21 | 1750.2 | CCTCCC | 12 |
| 22 | 1783.2 | ACCCAC | 13 |
| 23 | 1814.2 | CCATCG | 14 |
| 24 | 1845.2 | CGCTGT | 15 |
| 25 | 1877.2 | AAAGTT | 16 |
| 26 | 1909.2 | ATTGGG | 17 |
| 27 | 1943.2 | GAAGGG | 18 |
| 28 | 2039.4 | CCCTCCC | 19 |
| 29 | 2072.4 | CCCACCA | 20 |
| 30 | 2103.4 | CACCGCT | 21 |
| 31 | 2134.4 | CCTGCGT | 22 |
| 32 | 2165.4 | TTAATAA | 23 |
| 33 | 2197.4 | GTGTTAA | 24 |
| 34 | 2229.4 | GGTTGGT | 25 |
| 35 | 2263.4 | GGTGGAG | 26 |
| 36 | 2343.6 | CTCCCTCC | 27 |
| 37 | 2376.6 | CACCCATC | 28 |
| 38 | 2407.6 | GCTCCTAC | 29 |
| 39 | 2438.6 | CCTGTCTG | 30 |
| 40 | 2469.6 | ATTATATA | 31 |
| 41 | 2501.6 | GGTATATT | 32 |
| 42 | 2533.6 | GGTTGGTT | 33 |
| 43 | 2567.6 | GGGTTGGA | 34 |
| 44 | 2601.6 | GGGAGAGG | 35 |
| 45 | 2656.8 | ACCTCTCCC | 36 |
| 46 | 2689.8 | AAACCTCCC | 37 |
| 47 | 2720.8 | ACATCTCCG | 38 |
| 48 | 2751.8 | ACGTGCTTC | 39 |
| 49 | 2782.8 | ATAAATTTA | 40 |
| 50 | 2814.8 | AATTAGTTG | 41 |
| 51 | 2846.8 | AGTTGGTTG | 42 |
| 52 | 2880.8 | AGGGTATGG | 43 |
| 53 | 2914.8 | AGAGGAGGG | 44 |
| 54 | 2979.0 | AAACCCTCCC | 45 |
| 55 | 3012.0 | AAACACCCAC | 46 |
| 56 | 3043.0 | AAACGCACTC | 47 |
| 57 | 3074.0 | AAACCGGTCT | 48 |
| 58 | 3106.0 | AAACATAGAT | 49 |
| 59 | 3138.0 | AAACTAGTGG | 50 |
| 60 | 3172.0 | AAACGGAAGG | 51 |
| 61 | 3203.0 | AAGGAGGTAG | 52 |
| 62 | 3235.0 | AGAGGGTGGG | 53 |

### Oligonucleotide Probes for Detection of Colombia and Landsberg SNPs

Oligonucleotide probes (5′-3′ orientation) were selected to discriminate the SNP alleles for each of the twelve SNP loci described in Example 2. Primer-binding regions are underlined, stuffer sequences are double underlined and the EcoRI restriction enzyme recognition sequence is underlined in bold. All common reverse primers are phosphorylated at the 5′ end (Table 3).

TABLE 3

Oligonucleotide probes for detection of Colombia and Landsberg SNPs.

[SEQ.
ID

| | | | |
|---|---|---|---|
| NO] | Code | Nucleotide sequence | Stuffer |

| | | | |
|---|---|---|---|
| | SCGSNP1: | | |
| 54 | SNPfwd001 (G allele): | CGCCAGGGTTTTCCCAGTCACGACCGAATTCACTTCAGGACTAGTCTATACCTTG | C |
| | | AG | |
| 55 | SNPfwd002 (A allele): | CGCCAGGGTTTTCCCAGTCACGACGGAATTCACTTCAGGACTAGTCTAATACCTTG | G |
| | | AA | |
| 56 | SNPrev001 (Common | Phosphate-CTATGTGAACCAAATTAAAGTTTATCCTGTGTGAAATTGTTATCCGCT | |
| | reverse SNP001): | | |
| | SGCSNP20: | | |
| 57 | SNPfwd003 (A-allele) | CGCCAGGGTTTTCCCAGTCACGACCCGAATTCCTGCTCTTTCCTCGCTAGCTTCAG | CC |
| | | A | |
| 58 | SNPfwd004 (C-allele) | CGCCAGGGTTTTCCCAGTCACGACTAGAATTCCTGCTCTTTCCTCGCTAGCTTCAG | TA |
| | | C | |
| 59 | SNPrev002 (common | Phosphate-AGATTCGGACCTTCTCTCATAATTCCTGTGTGAAATTGTTATCCGCT | |
| | reverse SNP20): | | |
| | SGCSNP27: | | |
| 60 | SNPfwd005 (T-allele) | CGCCAGGGTTTTCCCAGTCACGACGGGAATTCGAAGAGGAGAGTGGCTACGAAC | GG |
| | | TCT | |
| 61 | SNPfwd006 (G-allele) | CGCCAGGGTTTTCCCAGTCACGACCCCGAATTCGAAGAGGAGAGTGGCTACGAA | CCC |
| | | CTCG | |
| 62 | SNPrev003 (common | Phosphate-GCGATAACTGCTCTGTAGAAAGACTCCTGTGTGAAATTGTTATCCGCT | |
| | reverse SNP27) | | |
| | SGCSNP37: | | |
| 63 | SNPfwd007 (C-allele) | CGCCAGGGTTTTCCCAGTCACGACTACGAATTCAATCGGCCTAAGCAAGCTTGTT | TAC |
| | | TTC | |
| 64 | SNPfwd008 (G-allele) | CGCCAGGGTTTTCCCAGTCACGACTGTGAATTCAATCGGCCTAAGCAAGCTTGTT | TGT |
| | | TTG | |
| 65 | SNPrev004 (common | Phosphate-TGCTATTGATATCTCTGTGCAACTTCCTGTGTGAAATTGTTATCCGCT | |
| | reverse SNP37) | | |
| | SGCSNP39 | | |
| 66 | SNPfwd009 (T-allele) | CGCCAGGGTTTTCCCAGTCACGACAGGGAATTCGATCGGAAAGATATCGGAGCT | AGG |
| | | CCTT | |
| 67 | SNPfwd010 (C-allele) | CGCCAGGGTTTTCCCAGTCACGACCCTCGAATTCGAGATCGGAAAGATATCGGA | CCTC |
| | | GCTCCTC | |
| 68 | SNPrev005 (common | Phosphate-GTCGGTGTCAACCGATCCACGGCGTCCTGTGTGAAATTGTTATCCGCT | |
| | reverse SNP39) | | |
| | SGCSNP44: | | |
| 69 | SNPfwd011 (A-allele) | CGCCAGGGTTTTCCCAGTCACGACACCAGAATTCGAACTGGCATCAACAGGCCT | ACCA |
| | | CCAA | |
| 70 | SNPfwd012 (T-allele) | CGCCAGGGTTTTCCCAGTCACGACCATGGAATTCGAACTGGCATCAATCAGGCCT | CATG |
| | | CCAT | |
| 71 | SNPrev006 (common | Phosphate-CCTTAATGCAAGGGCTTATTACGTTCCTGTGTGAAATTGTTATCCGCT | |
| | reverse SNP44) | | |
| | SGCSNP55 | | |
| 72 | SNPfwd013 (C-allele | CGCCAGGGTTTTCCCAGTCACGACTTGGGAATTCGGACTCCAAGGTATTGTTAGG | TTGG |
| | | CGCC | |
| 73 | SNPfwd014 (A-allele) | CGCCAGGGTTTTCCCAGTCACGACGGAGGAATTCGGACTCCAAGGTATTGTTAG | GGAG |
| | | GCGCA | |
| 74 | SNPrev007 (common | Phosphate-AACCACCAAGATCAGTCTCATCTTTCCTGTGTGAAATTGTTATCCGCT | |
| | reverse SNP55) | | |
| | SGCSNP69 | | |
| 75 | SNPfwd015 (G-allele) | CGCCAGGGTTTTCCCAGTCACGACCTCCCGAATTCCATCTCTTGCGCCTTCTCAGT | CTCCC |
| | | GTTG | |
| 76 | SNPfwd016 (A-allele) | CGCCAGGGTTTTCCCAGTCACGACCCACAGAATTCCATCTCTTGCGCCTTCTCAG | CCACA |
| | | TGTTA | |
| 77 | SNPrev008 (common | Phosphate-TGACGTCCGTCGAAGAATAGGTAATCCTGTGTGAAATTGTTATCCGCT | |
| | reverse SNP69) | | |
| | SGCSNP119: | | |
| 78 | SNPfwd017 (A-allele) | CGCCAGGGTTTTCCCAGTCACGACTAGCCGAATTCAGTTTCAAAACCCATGACGC | TAGCC |
| | | TTCTA | |
| 79 | SNPfwd018 (T-allele) | CGCCAGGGTTTTCCCAGTCACGACCTGGTGAATTCAGTTTCAAAACCCATGACGC | CTGGT |
| | | TTCTT | |
| 80 | SNPrev009 (common | Phosphate-GTGATAGCTGAAAAGACCCATTCTTCCTGTGTGAAATTGTTATCCGCT | |
| | reverse SNP119) | | |
| | SGCSNP164: | | |
| 81 | SNPfwd019 (T-allele) | CGCCAGGGTTTTCCCAGTCACGACGATGAGAATTCATACTCCAATTGCTCAGGCA | GATGA |
| | | CAGTT | |
| 82 | SNPfwd020 (C-allele) | CGCCAGGGTTTTCCCAGTCACGACGGGTGGAATTCGAATACTCCAATTGCTCAGG | GGGTG |
| | | CACAGTC | |
| 83 | SNPrev010 (common | Phosphate-CTCCTTGTCCCACGAAGATAGTTCTCCTGTGTGAAATTGTTATCCGCT | |
| | reverse SNP164) | | |
| | SGCSNP209: | | |
| 84 | SNPfwd021 (C-allele) | CGCCAGGGTTTTCCCAGTCACGACCCTCCCGAATTCGTAGAGGCTCTAAACAGCT | CCTCCC |
| | | GCTTCC | |
| 85 | SNPfwd022 (G-allele) | CGCCAGGGTTTTCCCAGTCACGACACCCACGAATTCGTAGAGGCTCTAAACAGCT | ACCCAC |
| | | GCTTCG | |
| 86 | SNPrev011 (common | Phosphate-CTTGTTTATGCTAAGGGCCGGCTCTCCTGTGTGAAATTGTTATCCGCT | |
| | reverse SNP209) | | |
| | SGCSNP312: | | |
| 87 | SNPfwd023 (G-allele) | CGCCAGGGTTTTCCCAGTCACGACCCATCGGAATTCTAAGTCAGCTCCTAAGCTT | CCATCG |
| | | CCATCG | |
| 88 | SNPfwd024 (T-allele) | CGCCAGGGTTTTCCCAGTCACGACCGCTGTGAATTCTAAGTCAGCTCCTAAGCTT | CGCTGT |
| | | CCATCT | |
| 89 | SNPrev012 (common | Phosphate-AAGCCACTTCCTCCTGCTCAAGCGTCCTGTGTGAAATTGTTATCCGCT | |
| | reverse SNP312) | | |

All oligonucleotides were purchased from Sigma-Aldrich Chemie, Zwijndrecht, The Netherlands. The concentrations of all oligonucleotides were adjusted to 1 μM

### Example 4

### Design of the PCR Amplification Primers

The sequences of the primers used for PCR amplification were complementary to the PCR primer binding regions incorporated in the ligation probes described in Example 3. The sequences represent the so called M13 forward and M13 reverse primers and their sequence in 5′-3′ orientation are:

| | |
|---|---|
| M13 forward: | |
| biotin-CGCCAGGGTTTTCCCAGTCACGAC | [SEQ ID NO:90] |
| M13 reverse: | |
| AGCGGATAACAATTTCACACAGGA | [SEQ ID NO:91] |

The concentration of these oligonucleotides was adjusted to 50 ng/μl. The M13 forward primer is biotinylated at the 5′ end to facilitate purification of the single stranded informative portion of the amplification product (bottom strand) after digestion with EcoRI and denaturation of the detectable fragment.

### Example 5

### Buffers and Reagents

The composition of the buffers was as follows:

Hybridisation buffer (1×): 20 mM Tris-HCl pH 8.5, 5 mM MgCl₂, 100 mM KCl, 10 mM DTT, 1 mM NAD^{+,}

Ligation buffer (1×): 20 mM Tris-HCl pH 7.6, 25 mM Kac, 10 mM MgAc₂, 10 mM DTT, 1 mM NAD^{+,} 0.1% Triton-X100

PCR buffer (10×): 10×PCR buffer was obtained from Qiagen, Valencia, United States of America and was used as such. No additions were used in the PCR

### Example 6

### Ligation, Amplification and Digestion

Ligation Reactions:

Ligation reactions were carried out as follows: 100 ng genomic DNA (1 μl of 100 ng/μl) in 5 ul total volume was heat denatured by incubation for 5 minutes at 94° C. and cooled on ice. Next 4 fmol of each OLA forward and reverse probes for 10 SNP loci (SGCSNP1, SGCSNP20, SGCSNP27, SGCSNP37, SGCSNP39, SGCSNP44, SGCSNP55, SGCSNP69, SGCSNP119 and SGCSNP164, SGCSNP209 and SGCSNP312) described in Example 2 were added (30 oligonucleotides in total), and the mixture was incubated for 16 hours at 60° C. Next, 1 unit of Taq Ligase (New England BioLabs) was added and the mixture was incubated for 15 minutes at 60° C.

Next, the ligase was heat-inactivated by incubation for 5 minutes at 94° C. and stored at minus 20° C. until further use.

PCR Amplification:

PCR reactions mixture contained 10 μl ligation mixture, 1 μl each of 50 ng/μl M13 forward and reverse primer (as described in Example 4), 200 uM of each dNTP, 2.5 Units HotStarTaq Polymerase (Qiagen), 5 μl 10×PCR buffer in a total volume of 50 μl. Amplifications were carried out by thermal cycling in a Perkin Elmer 9700 thermocycler (Perkin Elmer Cetus, Foster City, United States of America), according to one of the following thermal cycling profiles:

Profile 1: Initial denaturation/enzyme activation 15 min at 94° C., followed by 35 cycles of: 30 sec at 4° C., 30 sec at 55° C., 1 min at 72° C., and a final extension of 2 min at 72° C., 4° C., forever;

or:

Profile 2: Initial denaturation/enzyme activation 15 min at 94° C., followed by 35 cycles of: 5 sec at 94° C., 5 sec at 55° C., 10 sec at 72° C., and a final extension of, 2 min at 72° C., 4° C., forever.

Digestion with Restriction Enzyme EcoRI:

Double stranded amplification products were digested by adding 10 Units EcoRI (NEB), to the amplification mixture contained in 1×PCR buffer and incubation for 30 minutes at 37° C.

### Example 7

### Purification of Amplified Connected Probes

Purification of the biotinylated fragments (i.e. the digested amplification products and residual unincorporated biotinylated M13 forward PCR primers) for MALDI-TOF analysis was carried out as described essentially in WO 01/49882 by using streptavidin-coated beads according to standard procedures. Finally, the purified mixture of biotinylated oligonucleotides were eluted in 10 μl water and spotted for MALDI-TOF analysis.

### Example 8

### Detection by Mass Spectrometry

The bottom strands (mixture of purified single stranded oligonucleotides consists of the following components, schematically shown below:

5′biotin-CGCCAGGGTTTTCCCAGTCACGAC[stuffer sequence]G-3′ The stuffer sequence is unique for a particular allele of a particular SNP locus within one sample and all stuffers within that sample have a different (unique) mass. For the Arabidopsis SNPs shown in Example 2 and the selected OLA probes shown in Example 3, the expected masses of each purified single stranded oligonucleotide is shown in Table 4. In the Table, the total mass is the sum of the constant biotinylated PCR M13 forward primer, the stuffer sequence with variable mass, and the deoxynucleoside triphosphate that remains after digesting the EcoRI site at G/AATTC. The sum of the masses of the M13 forward PCR primer sequence and the G nucleotide is the constant mass. This constant mass is equal to the expected mass in case a stuffer sequence with length would have been used.

TABLE 4
Overview of the masses of the bottom strands obtained after digestion with
restriction enzyme EcoRI.

| | | Stuffer | Stuffer | Constant | |
|---|---|---|---|---|---|
| SNP Name | Allele | sequence | Mass(1) | mass(2) | Total mass |
| SGCSNP164 | C | GGGTG | 1621.0 | 7925.32 | 9546.32 |
| SGCSNP164 | T | GATGA | 1589.0 | 7925.32 | 9514.32 |
| SGCSNP119 | T | TAGCC | 1525.0 | 7925.32 | 9450.32 |
| SGCSNP119 | A | CTGGT | 1556.0 | 7925.32 | 9481.32 |
| SGCSNP69 | A | CCACA | 1494.0 | 7925.32 | 9419.32 |
| SGCSNP69 | G | CTCCC | 1461.0 | 7925.32 | 9386.32 |
| SGCSNP55 | A | GGAG | 1300.8 | 7925.32 | 9226.12 |
| SGCSNP55 | C | TTGG | 1266.8 | 7925.32 | 9192.12 |
| SGCSNP44 | T | CATG | 1235.8 | 7925.32 | 9161.12 |
| SGCSNP44 | A | ACCA | 1204.8 | 7925.32 | 9130.12 |
| SGCSNP39 | C | CCTC | 1171.8 | 7925.32 | 9097.12 |
| SGCSNP39 | T | AGG | 971.6 | 7925.32 | 8896.92 |
| SGCSNP37 | G | TGT | 937.6 | 7925.32 | 8862.92 |
| SGCSNP37 | C | TAC | 906.6 | 7925.32 | 8831.92 |
| SGCSNP27 | G | CCC | 867.6 | 7925.32 | 8792.92 |
| SGCSNP27 | T | GG | 658.4 | 7925.32 | 8583.72 |
| SGCSNP20 | C | TA | 617.4 | 7925.32 | 8542.72 |
| SGCSNP20 | A | CC | 578.4 | 7925.32 | 8503.72 |
| SGCSNP312 | T | CGCTGT | 1845.2 | 7925.32 | 9770.52 |
| SGCSNP312 | G | CCATCG | 1814.2 | 7925.32 | 9739.52 |
| SGCSNP209 | G | ACCCAC | 1783.2 | 7925.32 | 9708.52 |
| SGCSNP209 | C | CCTCCC | 1750.2 | 7925.32 | 9675.52 |
| SGCSNP1 | A | G | 329.2 | 7925.32 | 8254.52 |
| SGCSNP1 | G | C | 289.2 | 7925.32 | 8214.52 |
| Notes: | | | | | |
| (1)The stuffer mass is calculated based on sequence according to: Mass (Dalton) = (# G * 329.2 + # A * 313.2 + # T * 304.2 + # C * 289.2). | | | | | |
| (2)The constant mass is the mass of the PCR M13 forward primer: (biotin-CGCCAGGGTTTTCCCAGTCACGAC) + residual G remaining after cleaving with EcoRI at G/AATTC: Mass of biotin group (C₁₀H₁₆N₂O₃S) is 244.32 Dalton (Sigma). Mass of PCR primer M13 forward sequence is: 6 × 329.2 + 4 × 313.2 + 5 × 304.2 + 9 × 289.2 = 1975.2 + 1252.8 + 1521 + 2602.8 = 7351.8 Dalton. Mass of residual G is 329.2 Dalton. | | | | | |
| # Thus, the constant mass of the biotinylated strands of the digested amplification mixture is 244.32 + 7351.8 + 329.2 = 7925.32 Dalton. | | | | | |

In case linkers are used between the biotin and the primers sequence, the mass of the linker is incorporated in the constant mass.
Detection of the digested amplification mixture on the MALDI-TOF is carried out essentially as described in WO 01/49882.

### Example 9

### Detection of SNPs in Colombia and Landsberg Erecta Samples

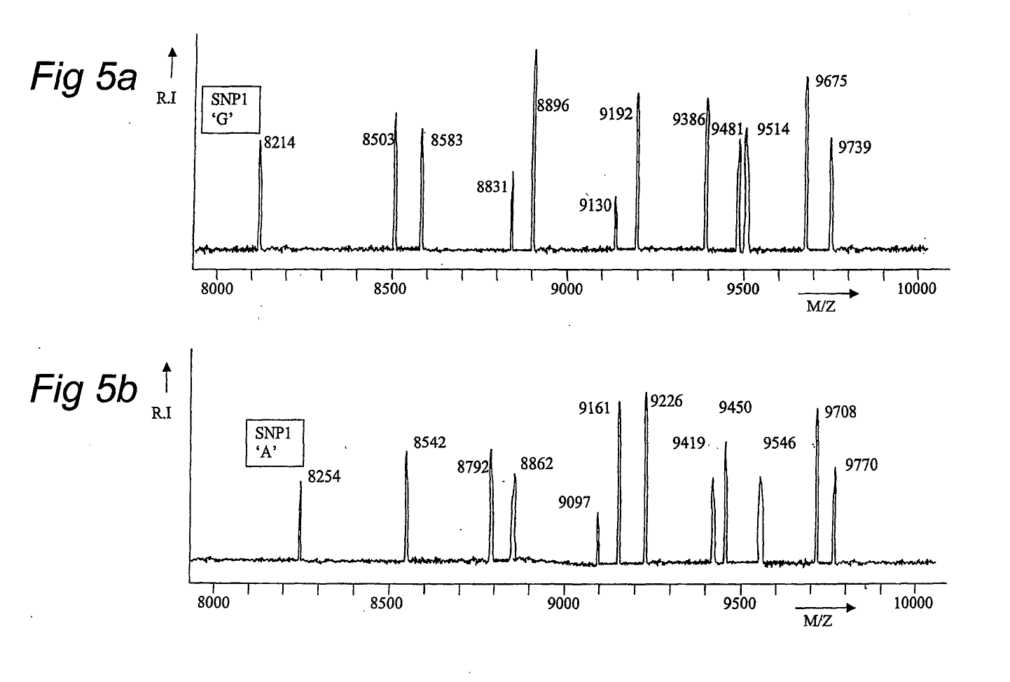

Mass spectrometric analysis of purified analytes (denatured detectable fragments, viz. bottom strands) of the SNP listed in Example 2 and prepared according to Examples 3-8. FIG. 5a shows the mass spectrum of the Colombia sample and FIG. 5b that of the Landsberg sample. It is clear that the appropriate alleles of the twelve SNP loci as defined in Example 2 and represented by analytes with a masses shown in Example 8 are observed. The peaks indicate that reliable genotyping of SNPs is achieved using this method.

### Example 10

### Suitable Stuffer Sequences

TABLE 4
Number of possible stuffer sequences in relation to maximum length and
mass difference. The maximum number of identical consecutive bases is 3
and the stuffer sequences do not contain an EcoRI site.

| | Number of possible stuffers | Number of possible stuffers |
|---|---|---|
| Maximum | sequences at 15 Dalton | sequences at 30 Dalton |
| stuffer length | resolution | resolution |
| 4 | 23 | 14 |
| 5 | 34 | 20 |
| 6 | 47 | 27 |
| 7 | 63 | 35 |
| 8 | 80 | 44 |
| 9 | 98 | 53 |
| 10 | 115 | 62 (see Example 3) |

TABLE 5
Number of possible stuffer sequences in relation to maximum length and
mass difference. The maximum number of identical consecutive bases is 3
and the stuffer sequences do not contain an BamHI site.

| | Number of possible stuffers | Number of possible stuffers |
|---|---|---|
| Maximum | sequences at 15 Dalton | sequences at 30 Dalton |
| stuffer length | resolution | resolution |
| 9 | 98 | 53 |
| 10 | 115 | 62 |

TABLE 6
Number of possible stuffer sequences in relation to maximum length and
mass difference. The maximum number of identical consecutive bases is 3
and the stuffer sequences do not contain an HindIII site.

| | Number of possible stuffers | Number of possible stuffers |
|---|---|---|
| Maximum | sequences at 15 Dalton | sequences at 30 Dalton |
| stuffer length | resolution | resolution |
| 9 | 98 | 53 |
| 10 | 115 | 62 |

From Tables 4, 5 and 6, it can be concluded that internal restriction enzyme sites for EcoRI, BamHI or HindIII are not likely to occur. Thus the choice of the restriction enzyme for digestion of amplification products does not significantly limit the number of possible stuffer sequences at a given mass resolution.

1. A method for determining the presence or absence of at least one target sequence (2) in a nucleic acid sample, comprising the steps of:
a) providing to a nucleic acid sample a pair of a first and a second oligonucleotide probe for each target sequence to be detected in the sample, whereby the first oligonucleotide probe has a section (4) at its 5′-end that is complementary to a first part (5) of a target sequence and the second oligonucleotide probe has a section (6) at its 3′-end that is complementary to a second part (7) of the target sequence, whereby the first (5) and second part (7) of the target sequence are located adjacent to each other, and whereby the first and second oligonucleotide probes (4, 6) each comprise a tag sequence (8, 9), whereby the tag sequences are essentially non-complementary to the target sequence, whereby the tag sequences comprise primer-binding sequences (12, 13), and wherein at least one of the tags further comprises a stuffer (11) and a restriction site (10) for a restriction enzyme, which restriction site (10) is located between the primer binding site and the section of the oligonucleotide probe (4, 6) that is complementary to the first (5) or second part (7) of the target sequence and wherein the stuffer (11) is located between the restriction site (10) and the primer binding site;
b) allowing the oligonucleotide probes to anneal to the adjacent parts of target sequence whereby the complementary sections (4,6) of the first and the second oligonucleotide probes are adjacent;
c) providing means (14) for connecting the first and the second oligonucleotide probes annealed adjacently to the target sequence and allowing the complementary sections (4, 6) of the adjacently annealed first and second oligonucleotide probes to be connected, to produce a connected probe (15) corresponding to a target sequence in the sample;
d) amplifying the connected probes from a primer pair (16, 17) to produce an amplified sample (19) comprising amplified connected probes (20);
e) digesting the amplified connected probes with the restriction enzyme to produce a detectable fragment (21);
f) detecting the presence or absence of the target sequence by detecting the presence or absence of the detectable fragment by a detection method based upon molecular mass.

2. Method according to claim 2, wherein a detectable fragment corresponding to a target sequence in a sample differs in mass from a detectable fragment corresponding to a different target sequence in the sample.

3. Method according to claim 2 or 3, wherein the detectable fragment is denatured to provide a top strand and a bottom strand.

4. Method according to claim 3, wherein the top strand is a single stranded oligonucleotide comprising the stuffer and wherein the bottom strand is essentially complementary to the top strand.

5. Method according to claim 3-4, wherein the top strand corresponding to a target sequence in a sample differs in mass from the top strand corresponding to a different target sequence in the sample.

6. Method according to claim 3-4, wherein the bottom strand corresponding to a target sequence in a sample differs in mass from the bottom strand corresponding to a different target sequence in the sample.

7. A method according to claim 2-5, wherein the difference in mass is provided by the mass of the stuffer in the top strand.

8. A method according to any one of claims 3-7, wherein the top strands and/or the bottom strands corresponding to different target sequences in the sample differ in mass by more than 1 Dalton.

9. A method according to claims 1-8, wherein a primer capable of annealing to the primer binding site in the detectable fragment comprises an affinity label.

10. A method according to claim 9, wherein the top strand and/or the bottom strand comprise the affinity label.

11. A method according to claim 9 or 10, wherein the detectable fragment, the top strand or the bottom strand is purified/isolated/separated from the sample comprising the amplified connected probes using the affinity label.

12. A method according to claim 9-11, wherein the affinity label is biotin.

13. A method according to claims 1-12, wherein the detection method is based on mass spectrometry, such as HPLC-MS, GC-MS, MALDI-TOF, ESI-MS.

14. A method according to claims 1-13, wherein the restriction enzyme is a restriction endonuclease.

15. A method according to claim 15, wherein the restriction endonuclease is a rare cutter.

16. A method according to claims 1-15, wherein a further mass difference between top strands corresponding to different target sequences is provided by incorporating different primer binding sites in the oligonucleotide probes to which different primers can anneal, preferably with a similar priming efficiency.

17. A method according to any one of the preceding claims, wherein the tag of the oligonucleotide probes comprise a stuffer sequence with a mass from 0 to 20000, preferably from 100 to 10000, more preferably from 500 to 5000.

18. A method according to any one of the preceding claims, wherein the presence or absence of at least 10, preferably at least 25, more preferably at least 50, still more preferably at least 100, most preferably at least 250 different target nucleotide sequences is determined in a nucleic acid sample.

19. A method according to any one of the preceding claims, wherein the length of the complementary section of the oligonucleotide probes is between 15 and 50 nucleotides, preferably between 18 and 40 nucleotides, more preferably between 20 and 30 nucleotides.

20. A method according to any one of the preceding claims, wherein the length of the primer-binding site is between 12 and 40 nucleotides, preferably between 15 and 30 nucleotides, more preferably between 17 and 25.

21. A method according to any one of the preceding claims, wherein the length of the tag is between 15 and 540 nucleotides, preferably between 18 and 140 nucleotides, more preferably between 20 and 75.

22. A method according to any one of the preceding claims, wherein the target nucleotide sequence contains a polymorphism, preferably a single nucleotide polymorphism.

23. A method according to any one of the preceding claims, wherein the target nucleotide sequence is a DNA molecule selected from the group consisting of: cDNA, genomic DNA, restriction fragments, adapter-ligated restriction fragments, amplified adapter-ligated restriction fragments and AFLP fragments.

24. A method according to any one of the preceding claims, further comprising a step for the removal of non-ligated probes, optionally prior to amplification, preferably by exonucleases.

25. A method according to any of the preceding claims wherein at least one of the primers is a selective primer.

26. A method according to claim 25, wherein the selective primer comprises a section that is complementary to at least part of the primer binding site and further contains a selective section of one to 10 selective nucleotides, preferably located immediately adjacent, to the 3′ end of the section complementary to the primer binding site.

27. A method according to claim 25 or 26 wherein the section that is complementary to at least part of the primer binding site preferably is complementary to 5, 10, 11, 12, 12, 14, 15, 16 or more nucleotides that form a part of the primer binding sequence that is located immediately adjacent, preferably at the 5′end, to the nucleotides complementary to the selective section of the primer.

28. Use of a method as defined in any of claims 1-27, for high throughput detection of a multiplicity of target nucleotide sequences.

29. Use of a method as defined in any of claims 1-27, for the detection of polymorphisms, preferably single nucleotide polymorphism.

30. Use of a method as defined in any of claims 1-27, for transcript profiling.

31. Use of a method as defined in any of claims 1-27, for the detection of the quantitative abundance of target nucleic acid sequences.

32. Use of a method as defined in any of claims 1-27, for genetic mapping, gene discovery, marker assisted selection, seed quality control, hybrid selection, QTL mapping, bulked segregant analysis, DNA fingerprinting and for disclosing information relating to traits, disease resistance, yield, hybrid vigor, and/or gene function.

33. A oligonucleotide acid probe for use in a method as defined in claims 1-27.

34. A set of two or more oligonucleotide probes, for use in a method as defined in claims 1-27.

35. Use of a set of two or more oligonucleotide probes as defined in claim 34, wherein the set comprises a probe for each allele of a single nucleotide polymorphism.

36. A set of primers for use in a method according to any one of claims 1-27.

37. A kit comprising oligonucleotide probes suitable for use in a method as defined in claims 1-27.

38. A kit comprising primers for use in a method as defined in claims 1-27.

39. A kit comprising primers and oligonucleotide probes for use in a method as defined in claims 1-27.


SEQUENCE LISTING (91 sequences)

SEQ ID NO: 1 - DNA, 4 bp, Artificial Sequence
  Description of Artificial Sequence stuffer sequence
cctc

SEQ ID NO: 2 - DNA, 4 bp, Artificial Sequence
  Description of Artificial Sequence stuffer sequence
acca

SEQ ID NO: 3 - DNA, 4 bp, Artificial Sequence
  Description of Artificial Sequence stuffer sequence
catg

SEQ ID NO: 4 - DNA, 4 bp, Artificial Sequence
  Description of Artificial Sequence stuffer sequence
ttgg

SEQ ID NO: 5 - DNA, 4 bp, Artificial Sequence
  Description of Artificial Sequence stuffer sequence
ggag

SEQ ID NO: 6 - DNA, 5 bp, Artificial Sequence
  Description of Artificial Sequence stuffer sequence
ctccc

SEQ ID NO: 7 - DNA, 5 bp, Artificial Sequence
  Description of Artificial Sequence stuffer sequence
ccaca

SEQ ID NO: 8 - DNA, 5 bp, Artificial Sequence
  Description of Artificial Sequence stuffer sequence
tagcc

SEQ ID NO: 9 - DNA, 5 bp, Artificial Sequence
  Description of Artificial Sequence stuffer sequence
ctggt

SEQ ID NO: 10 - DNA, 5 bp, Artificial Sequence
  Description of Artificial Sequence stuffer sequence
gatga

SEQ ID NO: 11 - DNA, 5 bp, Artificial Sequence
  Description of Artificial Sequence stuffer sequence
gggtg

SEQ ID NO: 12 - DNA, 6 bp, Artificial Sequence
  Description of Artificial Sequence stuffer sequence
cctccc

SEQ ID NO: 13 - DNA, 6 bp, Artificial Sequence
  Description of Artificial Sequence stuffer sequence
acccac

SEQ ID NO: 14 - DNA, 6 bp, Artificial Sequence
  Description of Artificial Sequence stuffer sequence
ccatcg

SEQ ID NO: 15 - DNA, 6 bp, Artificial Sequence
  Description of Artificial Sequence stuffer sequence
cgctgt

SEQ ID NO: 16 - DNA, 6 bp, Artificial Sequence
  Description of Artificial Sequence stuffer sequence
aaagtt

SEQ ID NO: 17 - DNA, 6 bp, Artificial Sequence
  Description of Artificial Sequence stuffer sequence
attggg

SEQ ID NO: 18 - DNA, 6 bp, Artificial Sequence
  Description of Artificial Sequence stuffer sequence
gaaggg

SEQ ID NO: 19 - DNA, 7 bp, Artificial Sequence
  Description of Artificial Sequence stuffer sequence
ccctccc

SEQ ID NO: 20 - DNA, 7 bp, Artificial Sequence
  Description of Artificial Sequence stuffer sequence
cccacca

SEQ ID NO: 21 - DNA, 7 bp, Artificial Sequence
  Description of Artificial Sequence stuffer sequence
caccgct

SEQ ID NO: 22 - DNA, 7 bp, Artificial Sequence
  Description of Artificial Sequence stuffer sequence
cctgcgt

SEQ ID NO: 23 - DNA, 7 bp, Artificial Sequence
  Description of Artificial Sequence stuffer sequence
ttaataa

SEQ ID NO: 24 - DNA, 7 bp, Artificial Sequence
  Description of Artificial Sequence stuffer sequence
gtgttaa

SEQ ID NO: 25 - DNA, 7 bp, Artificial Sequence
  Description of Artificial Sequence stuffer sequence
ggttggt

SEQ ID NO: 26 - DNA, 7 bp, Artificial Sequence
  Description of Artificial Sequence stuffer sequence
ggtggag

SEQ ID NO: 27 - DNA, 8 bp, Artificial Sequence
  Description of Artificial Sequence stuffer sequence
ctccctcc

SEQ ID NO: 28 - DNA, 8 bp, Artificial Sequence
  Description of Artificial Sequence stuffer sequence
cacccatc

SEQ ID NO: 29 - DNA, 8 bp, Artificial Sequence
  Description of Artificial Sequence stuffer sequence
gctcctac

SEQ ID NO: 30 - DNA, 8 bp, Artificial Sequence
  Description of Artificial Sequence stuffer sequence
cctgtctg

SEQ ID NO: 31 - DNA, 8 bp, Artificial Sequence
  Description of Artificial Sequence stuffer sequence
attatata

SEQ ID NO: 32 - DNA, 8 bp, Artificial Sequence
  Description of Artificial Sequence stuffer sequence
ggtatatt

SEQ ID NO: 33 - DNA, 8 bp, Artificial Sequence
  Description of Artificial Sequence stuffer sequence
ggttggtt

SEQ ID NO: 34 - DNA, 8 bp, Artificial Sequence
  Description of Artificial Sequence stuffer sequence
gggttgga

SEQ ID NO: 35 - DNA, 8 bp, Artificial Sequence
  Description of Artificial Sequence stuffer sequence
gggagagg

SEQ ID NO: 36 - DNA, 9 bp, Artificial Sequence
  Description of Artificial Sequence stuffer sequence
acctctccc

SEQ ID NO: 37 - DNA, 9 bp, Artificial Sequence
  Description of Artificial Sequence stuffer sequence
aaacctccc

SEQ ID NO: 38 - DNA, 9 bp, Artificial Sequence
  Description of Artificial Sequence stuffer sequence
acatctccg

SEQ ID NO: 39 - DNA, 9 bp, Artificial Sequence
  Description of Artificial Sequence stuffer sequence
acgtgcttc

SEQ ID NO: 40 - DNA, 9 bp, Artificial Sequence
  Description of Artificial Sequence stuffer sequence
ataaattta

SEQ ID NO: 41 - DNA, 9 bp, Artificial Sequence
  Description of Artificial Sequence stuffer sequence
aattagttg

SEQ ID NO: 42 - DNA, 9 bp, Artificial Sequence
  Description of Artificial Sequence stuffer sequence
agttggttg

SEQ ID NO: 43 - DNA, 9 bp, Artificial Sequence
  Description of Artificial Sequence stuffer sequence
agggtatgg

SEQ ID NO: 44 - DNA, 9 bp, Artificial Sequence
  Description of Artificial Sequence stuffer sequence
agaggaggg

SEQ ID NO: 45 - DNA, 10 bp, Artificial Sequence
  Description of Artificial Sequence stuffer sequence
aaaccctccc

SEQ ID NO: 46 - DNA, 10 bp, Artificial Sequence
  Description of Artificial Sequence stuffer sequence
aaacacccac

SEQ ID NO: 47 - DNA, 10 bp, Artificial Sequence
  Description of Artificial Sequence stuffer sequence
aaacgcactc

SEQ ID NO: 48 - DNA, 10 bp, Artificial Sequence
  Description of Artificial Sequence stuffer sequence
aaaccggtct

SEQ ID NO: 49 - DNA, 10 bp, Artificial Sequence
  Description of Artificial Sequence stuffer sequence
aaacatagat

SEQ ID NO: 50 - DNA, 10 bp, Artificial Sequence
  Description of Artificial Sequence stuffer sequence
aaactagtgg

SEQ ID NO: 51 - DNA, 10 bp, Artificial Sequence
  Description of Artificial Sequence stuffer sequence
aaacggaagg

SEQ ID NO: 52 - DNA, 10 bp, Artificial Sequence
  Description of Artificial Sequence stuffer sequence
aaggaggtag

SEQ ID NO: 53 - DNA, 10 bp, Artificial Sequence
  Description of Artificial Sequence stuffer sequence
agagggtggg

SEQ ID NO: 54 - DNA, 57 bp, Artificial Sequence
  Description of Artificial Sequence oligonucleotide probe
cgccagggttttcccagtcacgaccgaattcacttcaggactagtctataccttgag

SEQ ID NO: 55 - DNA, 57 bp, Artificial Sequence
  Description of Artificial Sequence oligonucleotide probe
cgccagggttttcccagtcacgacggaattcacttcaggactagtctataccttgaa

SEQ ID NO: 56 - DNA, 48 bp, Artificial Sequence
  Description of Artificial Sequence oligonucleotide probe
ctatgtgaaccaaattaaagtttatcctgtgtgaaattgttatccgct

SEQ ID NO: 57 - DNA, 57 bp, Artificial Sequence
  Description of Artificial Sequence oligonucleotide probe
cgccagggttttcccagtcacgacccgaattcctgctctttcctcgctagcttcaga

SEQ ID NO: 58 - DNA, 57 bp, Artificial Sequence
  Description of Artificial Sequence oligonucleotide probe
cgccagggttttcccagtcacgactagaattcctgctctttcctcgctagcttcagc

SEQ ID NO: 59 - DNA, 47 bp, Artificial Sequence
  Description of Artificial Sequence oligonucleotide probe
agattcggaccttctctcataattcctgtgtgaaattgttatccgct

SEQ ID NO: 60 - DNA, 57 bp, Artificial Sequence
  Description of Artificial Sequence oligonucleotide probe
cgccagggttttcccagtcacgacgggaattcgaagaggagagtggctacgaactct

SEQ ID NO: 61 - DNA, 58 bp, Artificial Sequence
  Description of Artificial Sequence oligonucleotide probe
cgccagggttttcccagtcacgaccccgaattcgaagaggagagtggctacgaactcg

SEQ ID NO: 62 - DNA, 48 bp, Artificial Sequence
  Description of Artificial Sequence oligonucleotide probe
gcgataactgctctgtagaaagactcctgtgtgaaattgttatccgct

SEQ ID NO: 63 - DNA, 58 bp, Artificial Sequence
  Description of Artificial Sequence oligonucleotide probe
cgccagggttttcccagtcacgactacgaattcaatcggcctaagcaagcttgttttc

SEQ ID NO: 64 - DNA, 58 bp, Artificial Sequence
  Description of Artificial Sequence oligonucleotide probe
cgccagggttttcccagtcacgactgtgaattcaatcggcctaagcaagcttgttttg

SEQ ID NO: 65 - DNA, 48 bp, Artificial Sequence
  Description of Artificial Sequence oligonucleotide probe
tgctattgatatctctgtgcaacttcctgtgtgaaattgttatccgct

SEQ ID NO: 66 - DNA, 58 bp, Artificial Sequence
  Description of Artificial Sequence oligonucleotide probe
cgccagggttttcccagtcacgacagggaattcgatcggaaagatatcggagctcctt

SEQ ID NO: 67 - DNA, 61 bp, Artificial Sequence
  Description of Artificial Sequence oligonucleotide probe
cgccagggttttcccagtcacgaccctcgaattcgagatcggaaagatatcggagctcctc

SEQ ID NO: 68 - DNA, 48 bp, Artificial Sequence
  Description of Artificial Sequence oligonucleotide probe
gtcggtgtcaaccgatccacggcgtcctgtgtgaaattgttatccgct

SEQ ID NO: 69 - DNA, 59 bp, Artificial Sequence
  Description of Artificial Sequence oligonucleotide probe
cgccagggttttcccagtcacgacaccagaattcgaactggcatcaatcaggcctccaa

SEQ ID NO: 70 - DNA, 59 bp, Artificial Sequence
  Description of Artificial Sequence oligonucleotide probe
cgccagggttttcccagtcacgaccatggaattcgaactggcatcaatcaggcctccat

SEQ ID NO: 71 - DNA, 48 bp, Artificial Sequence
  Description of Artificial Sequence oligonucleotide probe
ccttaatgcaagggcttattacgttcctgtgtgaaattgttatccgct

SEQ ID NO: 72 - DNA, 59 bp, Artificial Sequence
  Description of Artificial Sequence oligonucleotide probe
cgccagggttttcccagtcacgacttgggaattcggactccaaggtattgttaggcgcc

SEQ ID NO: 73 - DNA, 59 bp, Artificial Sequence
  Description of Artificial Sequence oligonucleotide probe
cgccagggttttcccagtcacgacggaggaattcggactccaaggtattgttaggcgca

SEQ ID NO: 74 - DNA, 48 bp, Artificial Sequence
  Description of Artificial Sequence oligonucleotide probe
aaccaccaagatcagtctcatctttcctgtgtgaaattgttatccgct

SEQ ID NO: 75 - DNA, 60 bp, Artificial Sequence
  Description of Artificial Sequence oligonucleotide probe
cgccagggttttcccagtcacgacctcccgaattccatctcttgcgccttctcagtgttg

SEQ ID NO: 76 - DNA, 60 bp, Artificial Sequence
  Description of Artificial Sequence oligonucleotide probe
cgccagggttttcccagtcacgacccacagaattccatctcttgcgccttctcagtgtta

SEQ ID NO: 77 - DNA, 48 bp, Artificial Sequence
  Description of Artificial Sequence oligonucleotide probe
tgacgtccgtcgaagaataggtaatcctgtgtgaaattgttatccgct

SEQ ID NO: 78 - DNA, 60 bp, Artificial Sequence
  Description of Artificial Sequence oligonucleotide probe
cgccagggttttcccagtcacgactagccgaattcagtttcaaaacccatgacgcttcta

SEQ ID NO: 79 - DNA, 60 bp, Artificial Sequence
  Description of Artificial Sequence oligonucleotide probe
cgccagggttttcccagtcacgacctggtgaattcagtttcaaaacccatgacgcttctt

SEQ ID NO: 80 - DNA, 48 bp, Artificial Sequence
  Description of Artificial Sequence oligonucleotide probe
gtgatagctgaaaagacccattcttcctgtgtgaaattgttatccgct

SEQ ID NO: 81 - DNA, 60 bp, Artificial Sequence
  Description of Artificial Sequence oligonucleotide probe
cgccagggttttcccagtcacgacgatgagaattcatactccaattgctcaggcacagtt

SEQ ID NO: 82 - DNA, 62 bp, Artificial Sequence
  Description of Artificial Sequence oligonucleotide probe
cgccagggttttcccagtcacgacgggtggaattcgaatactccaattgctcaggcacagtc

SEQ ID NO: 83 - DNA, 48 bp, Artificial Sequence
  Description of Artificial Sequence oligonucleotide probe
ctccttgtcccacgaagatagttctcctgtgtgaaattgttatccgct

SEQ ID NO: 84 - DNA, 61 bp, Artificial Sequence
  Description of Artificial Sequence oligonucleotide probe
cgccagggttttcccagtcacgaccctcccgaattcgtagaggctctaaacagctgcttcc

SEQ ID NO: 85 - DNA, 61 bp, Artificial Sequence
  Description of Artificial Sequence oligonucleotide probe
cgccagggttttcccagtcacgacacccacgaattcgtagaggctctaaacagctgcttcg

SEQ ID NO: 86 - DNA, 48 bp, Artificial Sequence
  Description of Artificial Sequence oligonucleotide probe
cttgtttatgctaagggccggctctcctgtgtgaaattgttatccgct

SEQ ID NO: 87 - DNA, 61 bp, Artificial Sequence
  Description of Artificial Sequence oligonucleotide probe
cgccagggttttcccagtcacgacccatcggaattctaagtcagctcctaagcttccatcg

SEQ ID NO: 88 - DNA, 61 bp, Artificial Sequence
  Description of Artificial Sequence oligonucleotide probe
cgccagggttttcccagtcacgaccgctgtgaattctaagtcagctcctaagcttccatct

SEQ ID NO: 89 - DNA, 48 bp, Artificial Sequence
  Description of Artificial Sequence oligonucleotide probe
aagccacttcctcctgctcaagcgtcctgtgtgaaattgttatccgct

SEQ ID NO: 90 - DNA, 24 bp, Artificial Sequence
  Description of Artificial Sequence PCR primer (M13 forward primer)
cgccagggttttcccagtcacgac

SEQ ID NO: 91 - DNA, 24 bp, Artificial Sequence
  Description of Artificial Sequence PCR primer (M13 reverse primer)
agcggataacaatttcacacagga